(19) United States
(12) Patent Application Publication
(10) Pub. No.: US 2023/0086782 A1
(43) Pub. Date: Mar. 23, 2023

(54) BASE EDITOR LACKING HNH AND USE THEREOF

(71) Applicant: ETH Zurich, Zurich (CH)

(72) Inventors: Gerald SCHWANK, Zürich (CH); Lukas VILLIGER, Zürich (CH)

(21) Appl. No.: 17/795,316

(22) PCT Filed: Jan. 20, 2021
(86) PCT No.: PCT/EP2021/051192

§ 371 Date: Jul. 26, 2022

(30) Foreign Application Priority Data
Jan. 27, 2020 (EP) 20153850.1

(51) Int. Cl. C12N 15/90; C12N 15/11; C12N 9/22; C12N 9/78; C12N 15/62

(52) U.S. Cl. CPC C12N 15/907; C12N 15/11; C12N 9/22; C12N 9/78; C12Y 305/04004; C12N 15/62; C12N 2310/20; C12N 2800/80; C12N 2800/107; C07K 2319/09

(57) ABSTRACT

The present invention relates to a chimeric enzyme comprising a CRISPR class 2 type II enzyme backbone, wherein the HNH domain in the backbone has been replaced, essentially, by a peptide or protein domain having catalytic activity on a single stranded polynucleotide.

### FIELD OF THE INVENTION

The present application relates to base editors and methods of editing a nucleobase or reversing a single nucleotide polymorphism.

### BACKGROUND

Base editing is a new genome editing technology that enables the direct, irreversible conversion of a specific DNA base into another at a targeted genomic locus. Importantly, this can be achieved without requiring double-stranded DNA breaks (DSB). Since many genetic diseases arise from point mutations, this technology has important implications in the study of human health and disease.

The first DNA base editors convert a CG base pair to a TA base pair by deaminating the exocyclic amine of the target cytosine to generate uracil (cytidine base editor, abbreviated CBE). To localize deamination activity to a small target window within the mammalian genome, Liu and coworkers used an APOBEC1 cytidine deaminase, which accepts ssDNA as a substrate but is incapable of acting on dsDNA. Fusion of APOBEC1 to the N-terminus of inactive Cas9 from Streptococcus pyogenes (“dCas9”, a mutant of Cas9 containing D1OA and H840A) resulted in a first base (Komor et al 2016). When bound to its cognate DNA, dCas9 performs local denaturation of the DNA duplex to generate an R-loop in which the DNA strand not paired with the guide RNA exists as a disordered single-stranded bubble. This feature enables the base editor to perform efficient and localized cytosine deamination in a test tube, with deamination activity restricted to a −5-bp window of ssDNA (positions −4−8, counting the protospacer adjacent motif (PAM) as positions 21-23) generated by dCas9. Fusion to dCas9 presents the target site to APOBEC1 in high effective molarity.

A major challenge for the use of base editors in mammalian cells is circumventing DNA repair processes that oppose target base pair conversion. One such mechanism is cellular repair of the UG intermediate in DNA. Base excision repair (BER) of UG in DNA is initiated by uracil N-glycosylase (UNG), which recognizes the UG mismatch and cleaves the glyosidic bond between uracil and the deoxyribose backbone of DNA. To inhibit UNG, Liu and co-workers fused uracil DNA glycosylase inhibitor (UGI), a small protein from bacteriophage PBS, to the C-terminus of the CBE. UGI is a DNA mimic that potently inhibits both human and bacterial UNG, hence enabling conversion of a CG base pair to a TA base pair through a UG intermediate

Later, Adenine base editors (ABEs) were developed, which are capable of converting an AT base pair into a GC base pair. ABEs are of particular interest because they enable correction of the most common type of pathogenic SNPs in the ClinVar database, representing −47% of disease-associated point mutation (Rees and Liu 2018). The major hurdle to the development of an ABE was the lack of any known adenosine deaminase enzymes capable of acting on ssDNA. To overcome this problem, Liu and co-workers evolved a deoxyadenosine deaminase enzyme that accepts ssDNA starting from an Escherichia coli tRNA adenosine deaminase enzyme, TadA (Gaudelli et al, 2017). Again, the deaminase was fused to the N-terminus of a dCas9.

Collectively, CBEs and ABEs can mediate all four possible transition mutations (C to T, A to G, T to C, and G to A. They have hence enormous promise for targeting disease-causing singe base pair mutations.

However, the way how these deaminase and Cas proteins were engineered remained the same - leading to largely identical activity windows, where only a small part within the R-Loop can be deaminated. In short, current base editors can only mutate some targets but not others.

All currently published approaches have similar editing windows, all of which are relatively narrow. Hence, there are still quite a few DNA loci which cannot be targeted with the currently available base editors, leaving e.g. many disease causing SNPs unadressable.

There is hence the need to provide base editors and base editing methods which have altered editing windows. There is further the need to provide base editors which allow targeting of DNA loci or SNPs which so far have not been addressable.

These and further objects are met with methods and means according to the independent claims of the present invention. The dependent claims are related to specific embodiments.

### SUMMARY OF THE INVENTION

The present invention provides a chimeric enzyme comprising a CRISPR class 2 type II enzyme backbone, wherein the HNH domain in the backbone has been replaced, essentially, by a peptide or protein domain having catalytic activity on a single stranded polynucleotide. The invention and general advantages of its features and embodiments will be discussed in detail below

### BRIEF DESCRIPTION OF THE FIGURES

The following terms are being used to describe the different constructs used herein. Note that these terms only describe the general domain structure, not the specific fusion peptides. The sequence given in the table are hence only exemplary and should not be construed as limiting.

| | Core Domain | Example |
|---|---|---|
| Name | structure (generalized) | sequence |
| ABEmax | Deaminase-RuvC1-REC- | 21 |
| | RuvC2-HNH-PuvC3-PI | |
| ABEmaxPI_1 | RuvC1-REC- | 18 |
| | RuvC2-HNH-PuvC3-PI | |
| | with integrated deaminase | |
| ABEmaxPI_2 | RuvC1-REC- | 19 |
| | RuvC2-HNH-PuvC3-PI | |
| | with integrated deaminase | |
| ABEmaxPI_3 | RuvC1-REC- | 20 |
| | RuvC2-HNH-PuvC3-PI | |
| | with integrated deaminase | |
| dABEmax | ABEmax lacking cleavage | 22 |
| | activity due to mutation in | |
| | HNH (e.g., H840A) | |
| ABEmax | ABEmax lacking the | 23 |
| ΔHNH | HNH domain | |
| HNHx ABE | RuvC1-REC-RuvC2- | 12 |
| | Deaminase-RuvC3-PI | |

FIG. 1: Concepts to increase substrate accessiblilty for base editing at PAM-proximal bases.
a) Schematic domain organization of ABEmax and ABEmax PI1-3. ABEmax PI1-3 comprise an SpCas9 (D10A), where the TadA deaminase is integrated within the PI domain. ABEmax PI1, PI2, and PI3 use different linker lengths flanking the TadA deaminase.
b) Editing efficiencies of ABEmax PI constructs are shown as mean±s.d. at 3 different sites.
c) Structural data of hypothetical base editors with and without HNH domain.
d) Schematic domain organization of ABEmax-AHNH.
e) Effects of reducing nickase activity of ABEmax base editors to catalytically dead ABEmax with (dABEmax) and without (ABEmax MINH) HNH domain.

RECTIFIED SHEET (RULE 91) ISA/EP

FIG. 2: HNH domain substitution with sfGPF and deaminase domains
a) Schematic domain organization of HNHx ABE (shown as HNHx TadA ABE).
b) Structural data of hypothetical Cas9 constructs, where the HNH domain is replaced by sfGFP or a TadA deaminase.
c) Fluorescence microscopy of HEK293T expressing Cas9, where the HNH domain is replaced with sfGFP with and without nuclear localization signals.
d) Heatmap depicting different linkers to incorporate the TadA deaminase in place of the HNH domain.

FIG. 3: Targeting of endogenous adenine bases

Editing efficiencies of different adenine bases within the protospacer region of ABEmax and HNHx ABE. Numbering starts with the PAM-distal nucleotides. Data represent mean±s.d.

FIG. 4. Reaction scheme of base editing mechanisms

Scheme shows basic reaction of cytidine base editing an adenine base editing. Hydrolytic deamination of cytosine (C) by deaminases generates uridine as a product. Hydrolytic deamination of adenosine (A) generates Inosine as a product Uridine and Inosine are read as thymine (T) and guanosine (G) by the cellular machinery, e.g., by polymerase enzymes.

FIG. 5: Molecular structure of HNHx ABE

Removal of HNH and replacement by deaminase gives access to ssDNA.

FIG. 6. Heat map depicting editing efficiencies of different constructs incorporating the PmCDA1 deaminase in place of the HNH domain.

Different linkers are used to incorporate the PmCDA1 deaminase in place of the HNH domain and editing efficiencies read out by high throughput sequencing.

FIG. 7. Heat map depicting editing efficiencies of different constructs incorporating the FERNY deaminase in place of the HNH domain.

Different linkers are used to incorporate the FERNY deaminase in place of the HNH domain into editing efficiencies read out by high throughput sequencing.

### DETAILED DESCRIPTION OF THE INVENTION

Before the invention is described in detail, it is to be understood that this invention is not limited to the particular component parts of the devices described or process steps of the methods described as such devices and methods may vary. It is also to be understood that the terminology used herein is for purposes of describing particular embodiments only, and is not intended to be limiting. It must be noted that, as used in the specification and the appended claims, the singular forms “a”, “an”, and “the” include singular and/or plural referents unless the context clearly dictates otherwise. It is moreover to be understood that, in case parameter ranges are given which are delimited by numeric values, the ranges are deemed to include these limitation values.

It is further to be understood that embodiments disclosed herein are not meant to be understood as individual embodiments which would not relate to one another. Features discussed with one embodiment are meant to be disclosed also in connection with other embodiments shown herein. If, in one case, a specific feature is not disclosed with one embodiment, but with another, the skilled person would understand that does not necessarily mean that said feature is not meant to be disclosed with said other embodiment. The skilled person would understand that it is the gist of this application to disclose said feature also for the other embodiment, but that just for purposes of clarity and to keep the specification in a manageable volume this has not been done.

Furthermore, the content of the prior art documents referred to herein is incorporated by reference. This refers, particularly, for prior art documents that disclose standard or routine methods. In that case, the incorporation by reference has mainly the purpose to provide sufficient enabling disclosure, and avoid lengthy repetitions.

According to a first aspect of the invention, a chimeric enzyme comprising a CRISPR class 2 type II enzyme backbone is provided, wherein the HNH domain in the backbone has been replaced, essentially, by a peptide or protein domain having catalytic activity on a single stranded polynucleotide.

As used herein, the term “domain A has been replaced, essentially, by another domain” means that a given domain within a protein, or at least a functional fragment thereof, has been removed and replaced by another domain, or at least a functional fragment thereof. In such way, the protein loses the functionality of the first domain, and also loses at least part of the peptide stretch of the first domain. The new domain or the functional fragment that is inserted (or appended, depending on the position of the first domain in the protein) can be flanked by one or two linkers.

As used herein, the term “CRISPR class 2 type II enzyme” refers to an enzyme which is capable to bind to double stranded nucleotides and, as wildtype, has both the RuvC and HNH nuclease. Generally, the domain structure of CRISPR class 2 type II enzyme enzymes is as follows (N->C):

RuvC-I— Recognition lobe—RuvC-II— HNH— RuvC-III— PI

Therein, PI refers to the PAM-interacting domain, whereas the recognition lobe harbors the crRNA and tracrRNA, or the single guide RNA.

In one embodiment, the domain structure of such chimeric enzyme is as follows:

RuvC-I— Recognition lobe—RuvC-II— PEPTIDE— RuvC-III— PI wherein “PEPTIDE” refers to the peptide or protein domain having catalytic activity on a single stranded polynucleotide.

As used herein, the term “peptide or protein domain having catalytic activity on a single stranded polynucleotide” refers to enzymatic entities which are capable of (i) cleaving, chemically modifying, transcribing, translating, or transposing individual nucleotides in a single stranded polynucleotide or
(ii) cleaving, chemically modifying, transcribing, translating, or transposing a polynucleotide stretch within a single stranded polynucleotide or

According to one embodiment, the peptide or protein domain having catalytic activity on a single stranded nucleotide is a peptide or protein domain having at least one selected from the group consisting of
a) deaminase activity,
b) reverse transcriptase activity,
c) methyltransferase activity,
d) transposase activity,
e) polymerase activity, and
f) nuclease activity

In the following, a peptide or protein domain having deaminase activity will also be called “deaminase” herein, while a peptide or protein domain having reverse transcriptase activity will also be called “reverse transcriptase” herein.

A peptide or protein domain having methyltransferase activity, will also be called “methyltransferase” herein, a peptide or protein domain having transposase activity, will also be called “transposase” herein, a peptide or protein domain having polymerase activity, will also be called “polymerase” herein, and a peptide or protein domain having nuclease activity will also be called “nuclease” herein.

By replacing the HNH domain by a deaminase, the domain structure of the chimeric enzyme according to the present invention comprises at least the following elements:

RuvC-I— Recognition lobe—RuvC-II— deaminase—RuvC-III— PI or RuvC-I— Recognition lobe—RuvC-II— reverse transcriptase—RuvC-III— PI or RuvC-I— Recognition lobe—RuvC-II— methyltransferase—RuvC-III— PI or RuvC-I— Recognition lobe—RuvC-II— transposase—RuvC-III— PI or RuvC-I— Recognition lobe—RuvC-II— polymerase—RuvC-III— PI or RuvC-I— Recognition lobe—RuvC-II— nuclease—RuvC-III— PI

Hence, the domain structure of the chimeric enzyme according to the present invention is markedly different from other CRISPR-related base editing enzymes, like base editing, where the base editing enzyme is fused to the N-terminus of the enzyme backbone, and the HNH domain remains in the backbone (yet is sometimes silenced by including respective substitutions, like H840, H868, N882 and N891). Reference is made, e.g., to Kim et al (2017), the content of which is incorporated herein by reference, where the domain structure of the given base editor is disclosed as follows (N->C)

Deaminase - RuvC-I - Recognition lobe - RuvC-II— HNH - RuvC-III— PI with optionally NLS sequences at the N- and/or C terminus. Other configurations are disclosed in a more simplified fashion in US20190225955A1, paragraph [0162]:

NLS-Cas9-Deaminase NLS-Deaminase-Cas9 Cas9-NLS-Deaminase Deaminase-NLS-Cas9 Deaminase-Cas9-NLS Cas9-deaminase-NLS

All these embodiments have in common that the deaminase is attached to the entire Cas9 either N- or C-terminally. Other such base editors are disclosed, inter alia, in Gaudelli et al. 2018 and Komor et al. 2016, the contents of which are incorporated herein by reference.

In prime editing, the reverse transcriptase is attached to the C terminus of the enzyme backbone, and, likewise, the HNH domain remains in the backbone (yet is sometimes silenced by including respective substitutions, like H840, H868, N882 and N891). Reference is made, e.g., to Anzalone et al (2019), the content of which is incorporated herein by reference where the domain structure of the given prime editor is disclosed as follows (N->C):

RuvC-I - Recognition lobe - RuvC-II— HNH - RuvC-III— PI— Reverse Transcriptase

According to one embodiment, the CRISPR class 2 type II enzyme backbone is a CRISPR Cas9 enzyme backbone.

According to one embodiment, the CRISPR Cas9 enzyme backbone is a backbone taken from one member of the group consisting of SaCas9, SpCas9, StCas9, CjCas9, and NmeCas9.

SaCas9 is a Cas9 enzyme from Staphylococcus aureus (UniProtKB—J7RUA5(CAS9 STAAU)) SpCas9 (sometimes also called SpyCas9) is a Cas9 enzyme from Streptococcus pyogenes (UniProtKB—Q99ZW2 (CAS9 STRP1)). StCas9 is a Cas9 enzyme from Streptococcus thermophilus (UniProtKB—G3ECR1 (CAS9 STRTR). CjCas9 is a Cas9 enzyme from Campylobacter jejuni (UniProtKB—Q0P897 (CAS9 CAMJE). NmeCas9 is a Cas9 enzyme from Neisseria meningitidis (UniProtKB—A1IQ68 (CAS9 NEIMA)

According to one embodiment, the CRISPR Cas9 enzyme backbone (prior to replacement of the HNH domain) comprises
a) an amino acid sequence set forth in SEQ ID NO 1, SEQ ID NO 2, SEQ ID NO 3, SEQ ID NO 16 or SEQ ID NO 17, or
b) an amino acid sequence having at least 80% sequence identity therewith.

Both embodiments refer to the original backbone sequence prior to replacement of the HNH domain.

In some embodiments, the CRISPR Cas9 enzyme backbone comprises an amino acid sequence that has >81%, preferably >82%, more preferably >83%, >84%, >85%, >86%, >87%, >88%, >89%, >90%, >91%, >92%, >93%, >94%, >95%, >96%, >97%, >98 or most preferably >99% sequence identity with SEQ ID NO 1, SEQ ID NO 2, SEQ ID NO 3, SEQ ID NO 16 or SEQ ID NO 17 (i.e., prior to replacement of the HNH domain).

“Percentage of sequence identity” as used herein, is determined by comparing two optimally aligned sequences over a comparison window, wherein the portion of the polynucleotide sequence in the comparison window may comprise additions or deletions (i.e., gaps) as compared to the reference sequence (e.g., a polypeptide), which does not comprise additions or deletions, for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

In some embodiments, the CRISPR Cas9 enzyme backbone comprises an amino acid sequence set forth as above, with the proviso that it comprises at least one amino acid substitution which is a conservative amino acid substitution.

A “conservative amino acid substitution”, as used herein, has a smaller effect on enzyme function than a non-conservative substitution. Although there are many ways to classify amino acids, they are often sorted into six main groups on the basis of their structure and the general chemical characteristics of their R groups.

In some embodiments, a “conservative amino acid substitution” is one in which the amino acid residue is replaced with an amino acid residue having a similar side chain. For example, families of amino acid residues having similar side chains have been defined in the art. These families include amino acids with
basic side chains (e.g., lysine, arginine, histidine),
acidic side chains (e.g., aspartic acid, glutamic acid),
uncharged polar side chains (e.g., glycine, asparagine, glutamine, serine, threonine, tyrosine, cysteine),
nonpolar side chains (e.g., alanine, valine, leucine, isoleucine, proline, phenylalanine, methionine, tryptophan),
beta-branched side chains (e.g., threonine, valine, isoleucine) and aromatic side chains (e.g., tyrosine, phenylalanine, tryptophan, histidine).

Other conserved amino acid substitutions can also occur across amino acid side chain families, such as when substituting an asparagine for aspartic acid in order to modify the charge of a peptide. Conservative changes can further include substitution of chemically homologous non-natural amino acids (i.e., a synthetic non-natural hydrophobic amino acid in place of leucine, a synthetic non-natural aromatic amino acid in place of tryptophan).

It is vital that, in one embodiment, all enzymes falling under the above scope maintain their target sequence recognition capacity, i.e., dysfunctional variants are excluded from scope.

According to one embodiment, the CRISPR Cas9 enzyme backbone is catalytically inactive and/or lacks endonuclease activity.

In this context, the Cas9 enzyme backbone can comprise mutation(s) in the catalytic residues of either the RuvC-like domains (while the HNH domain is lacking). As non-limiting example, the catalytic residues of the compact Cas9 protein can comprise a substitution or deletion at least one position selected from D8, D10, D14, D16, D30 or D31 of any of SEQ ID NO 1-3, 16 and 17, where applicable, or at aligned positions using the CLUSTALW method on homologues of Cas9 family members. Any of these residues can be replaced by any other amino acids, preferably by alanine residue.

For example, a typical mutation silencing the RuvC domain in SpCas and SaCas9 is a mutation at D10, like, e.g. D1OA. In CjCas9, the corresponding mutation is at D8, e.g., D8A, while in NmeCas9 the corresponding mutation is at D16, e.g., D16A.

According to one embodiment, the deaminase catalyzes
a) deamination of cytosine, or
b) deamination of adenosine.

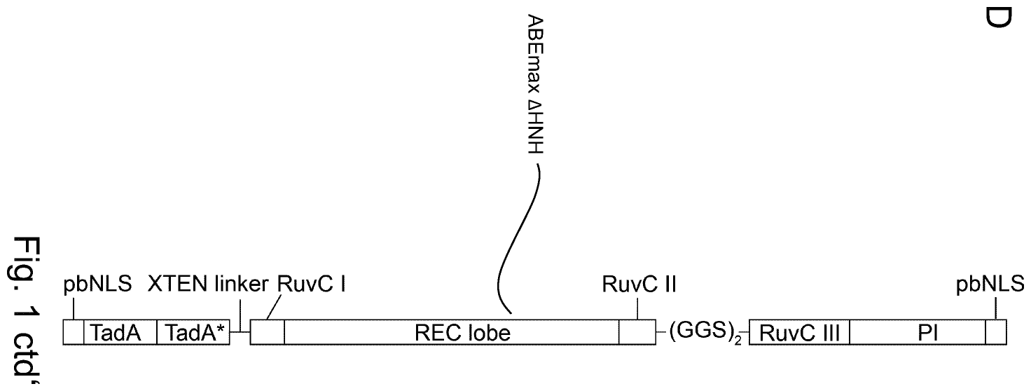

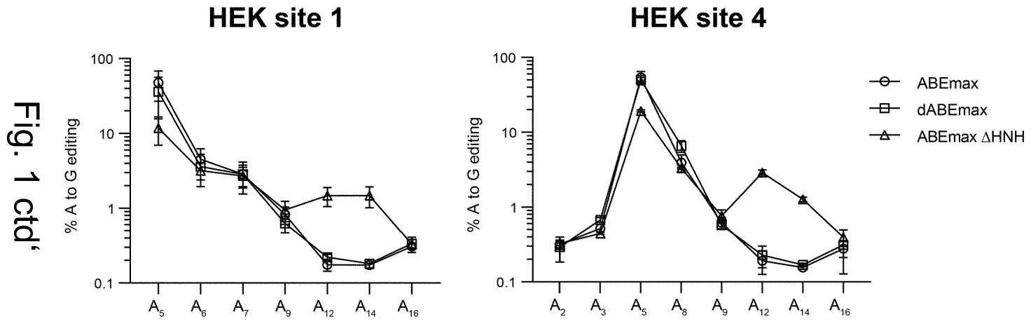

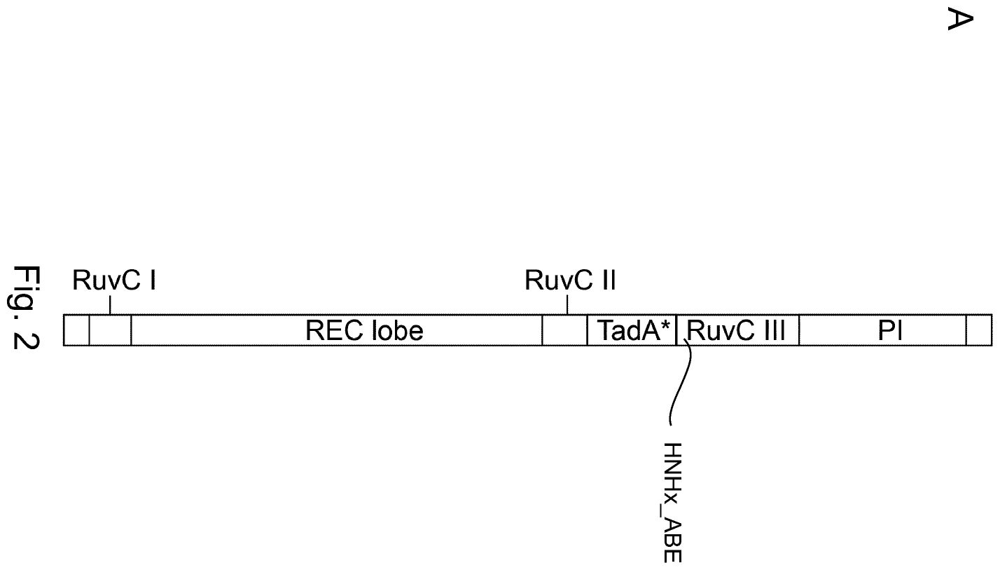

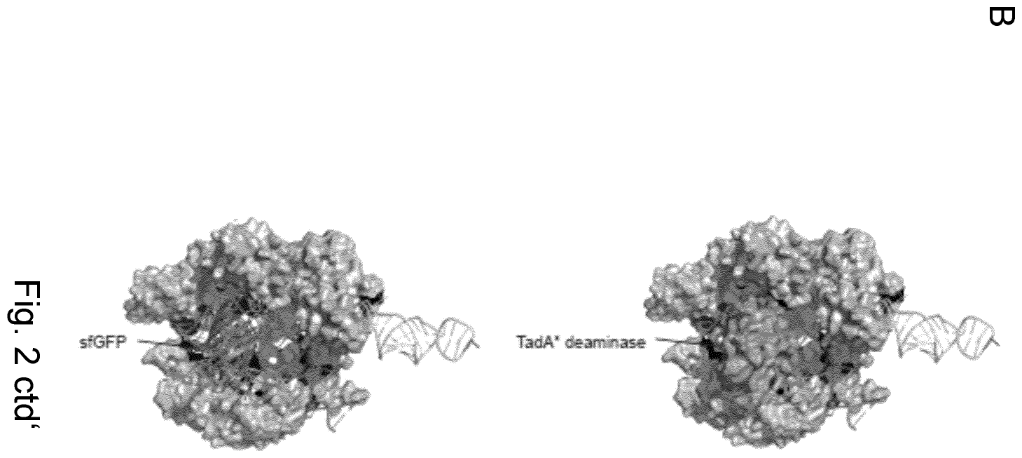

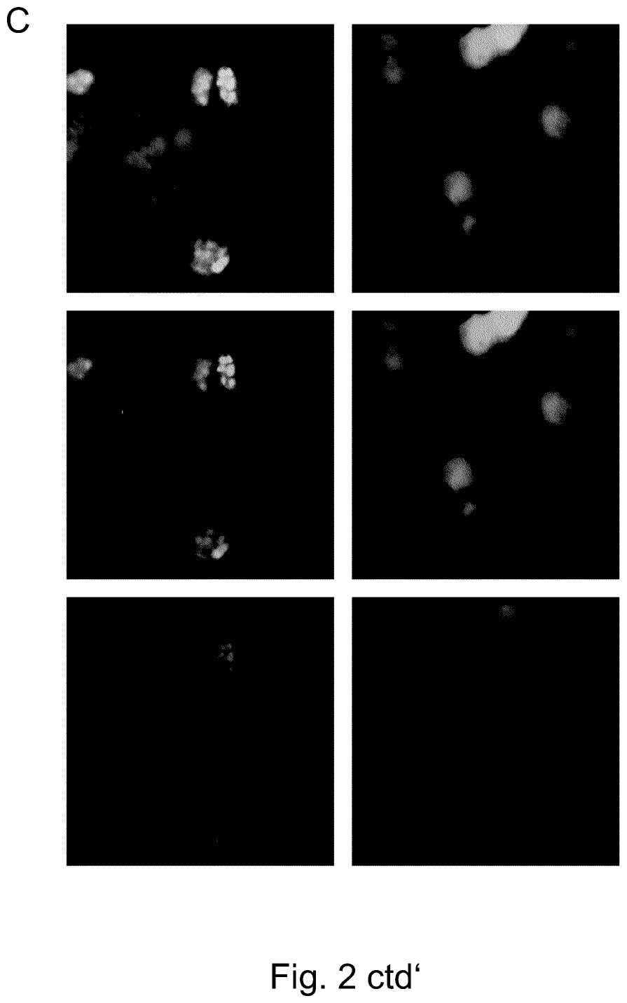

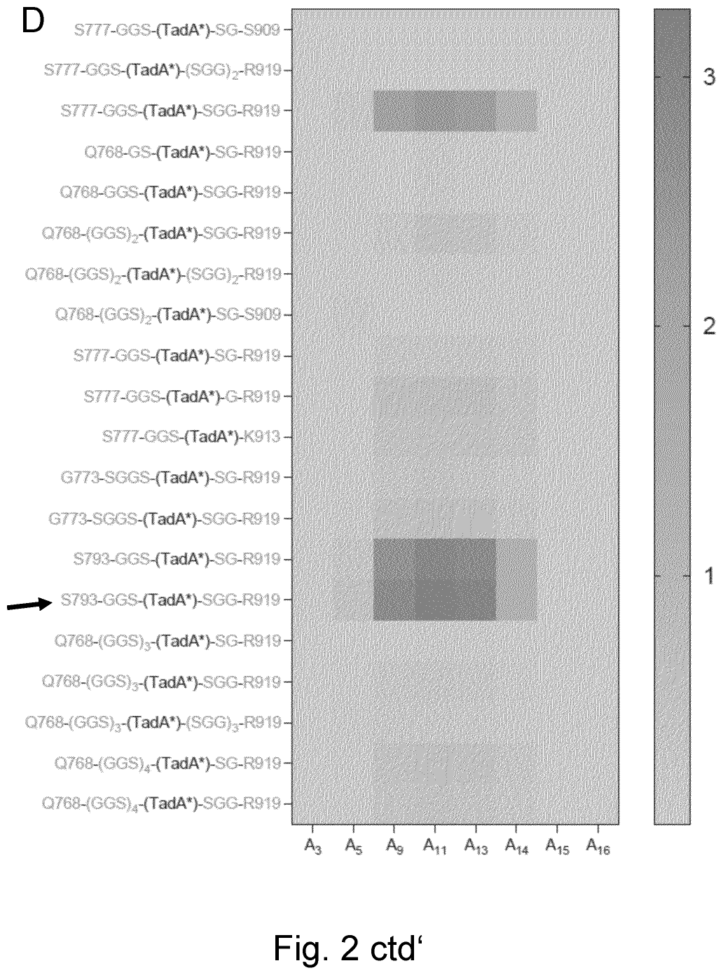

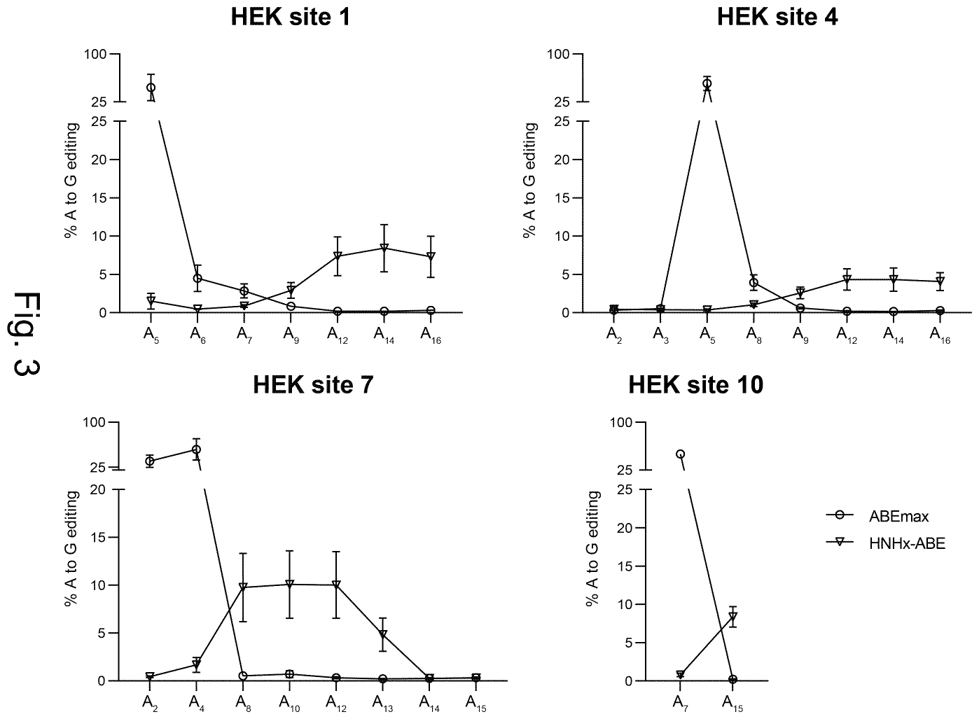

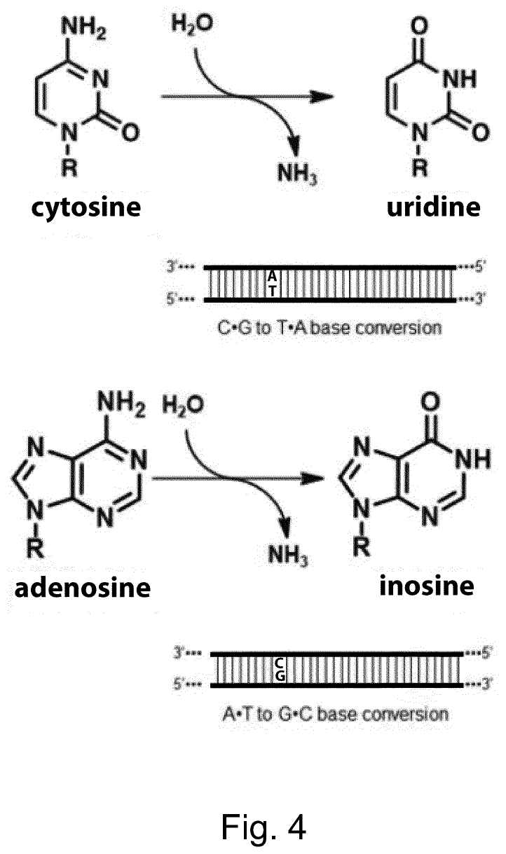

The reaction schemes of these two reactions are shown in FIG. 4. In case of a) the enzyme is called a Cytosine base editor (CBE), while in case of b) the enzyme is called an Adenosine base editor (ABE).

According to one embodiment, the deaminase comprises at least one of the enzymes selected from the group consisting of apolipoprotein B mRNA-editing complex (APOBEC) deaminase cytidine deaminase, and/or adenosine deaminase or at least a catalytically active domain derived therefrom maintaining deaminase activity. The following table shows more details of these deaminases:

| | | Source (UniProt | SEQ |
|---|---|---|---|
| | example | or literature | ID |
| class | deaminase | reference) | NO |
| apolipoprotein B | Apobec1 | P41238 | 5 |
| mRNA-editing | Ferny | Thuronyi et al | 7 |
| complex | PmCDA1 | A5H718 | 6 |
| CDA | | | |
| adenosine | TadA | P68398 from | 4 |
| deaminase | | E. coli | |

According to one embodiment, the deaminase comprises an amino acid sequence selected from a. the group consisting of enzymes selected from the group consisting of SEQ ID

NO 4-7, or b. a sequence having at least 80% sequence identity with SEQ ID NO 4-7 while maintaining deaminase activity, or c. a catalytically active domain derived from the deaminase of a) or b), with the optional proviso that SEQ ID NO 4 has at least one amino acid substitution selected from the group consisting of D108N, A106V, D147Y, E155V, L84F, H123Y, and/or I157F, and/or SEQ ID NO 6 has at least one amino acid substitution selected from the group consisting of F22S, A123V, and/or I195F.

In some embodiments, the deaminase comprises an amino acid sequence that has >81%, preferably >82%, more preferably >83%, >84%, >85%, >86%, >87%, >88%, >89%, >90%, >91%, >92%, >93%, >94%, >95%, >96%, >97%, >98 or most preferably >99% sequence identity with SEQ ID NO 4, SEQ ID NO 5, SEQ ID NO 6 or SEQ ID NO 7.

In some embodiments, the deaminase comprises an amino acid sequence set forth as above, with the proviso that it comprises at least one amino acid substitution which is a conservative amino acid substitution.

It is vital that, in one embodiment, all deaminases falling under the above scope maintain their deaminase activity, i.e., dysfunctional variants are excluded from scope.

Other suitable adenosine deaminase that can be used in the chimeric enzyme according to the invention are disclosed in U.S. Ser. No. 10/113,163, the content of which is incorporated herein, including the tRNA-specific adenosine (TadA) deaminases
Staphylococcus aureus TadA (SEQ ID NO 8 in U.S. Ser. No. 10/113,163)
Bacillus subtilis TadA (SEQ ID NO 9 in U.S. Ser. No. 10/113,163)
Salmonella typhimurium TadA (SEQ ID NO 9 in U.S. Ser. No. 10/113,163)
Shewanella putrefaciens TadA (SEQ ID NO 372 in U.S. Ser. No. 10/113,163
Caulobacter crescentus TadA (SEQ ID NO 374 in U.S. Ser. No. 10/113,163)
Haemophilus influenzae TadA (SEQ ID NO 373 in U.S. Ser. No. 10/113,163)
Geobacter sulfurreducens TadA (SEQ ID NO 375 in U.S. Ser. No. 10/113,163)

Other suitable apolipoprotein B mRNA-editing complex (APOBEC) deaminases that can be used in the chimeric enzyme according to the invention are disclosed in US20190225955A1 and WO2017070632, the content of which is incorporated herein, including
APOBEC2
APOBEC3
APOBEC3 A
APOBEC3D
APOBEC3E
APOBEC3F
APOBEC3G (SEQ ID NO 275 or 5739-5741 in WO2017070632)
APOBEC3H
APOBEC4

Other suitable deaminases that can be used in the chimeric enzyme according to the invention are disclosed in US20190225955A1, the content of which is incorporated herein, including
ACF1/ASE deaminase
ADAT family deaminase.

Other suitable cytidine deaminases that can be used in the chimeric enzyme according to the invention are disclosed in WO2017070632, the content of which is incorporated herein, including
Activation-Induced Cytosine Deaminase (AID) (SEQ ID NO 586 in WO2017070632)
Human AID-DC (truncated version of hAID with 7-fold increased activity) (SEQ ID NO: 608 in WO2017070632)

According to one embodiment, the reverse transcriptase comprises M-MLV RT (Moloney Murine Leukemia Virus Reverse Transcriptase) or at least a catalytically active domain derived therefrom maintaining reverse transcriptase activity.

According to one embodiment, the reverse transcriptase comprises an amino acid sequence selected from
a) SEQ ID NO 15, or
b) a sequence having at least 80% sequence identity with SEQ ID NO 15 while reverse transcriptase activity, or
c) a catalytically active domain derived from reverse transcriptase of a) or b), with the optional proviso that
SEQ ID NO 15 has at least one amino acid substitution selected from the group consisting of D200N, T306K, W313F, T330P, L603W

In some embodiments, the reverse transcriptase comprises an amino acid sequence that has >81%, preferably >82%, more preferably >83%, >84%, >85%, >86%, >87%, >88%, >89%, >90%, >91%, >92%, >93%, >94%, >95%, >96%, >97%, >98 or most preferably >99% sequence identity with SEQ ID NO 15.

In some embodiments, the reverse transcriptase comprises an amino acid sequence set forth as above, with the proviso that it comprises at least one amino acid substitution which is a conservative amino acid substitution.

It is vital that, in one embodiment, all reverse transcriptases falling under the above scope maintain their reverse transcriptase activity, i.e., dysfunctional variants are excluded from scope.

According to one embodiment, the enzyme further comprises
a) at least one nuclear localization sequence (NLS), and/or
b) at least one inhibitor of nucleic acid repair, preferably a Uracil-DNA glycosylase inhibitor (UGI)

The term “uracil glycosylase inhibitor” or “UGI,” as used herein, refers to a protein that is capable of inhibiting a uracil-DNA glycosylase base-excision repair enzyme.

The term “inhibitor of base repair” or “IBR” refers to a protein that is capable in inhibiting the activity of a nucleic acid repair enzyme, for example a base excision repair enzyme. In some embodiments, the IBR is an inhibitor of inosine base excision repair. Exemplary inhibitors of base repair include inhibitors of APE1, Endo III, Endo IV, Endo V, Endo VIII, Fpg, hOGG1, hNEILl, T7 Endol, T4PDG, UDG, hSMUG1, and hAAG. In some embodiments, the IBR is an inhibitor of Endo V or hAAG. In some embodiments, the IBR is a catalytically inactive EndoV or a catalytically inactive hAAG.

The nuclear localization sequence can be arranged at the N-terminus or the C terminus of the chimeric enzyme, or at both termini. The inhibitor of nucleic acid repair can be arranged at the C-terminus of the chimeric enzyme, preferably N-terminally of a n optional nuclear localization sequence, and preferably in duplicate.

Preferably, the nuclear localization sequence comprises an amino acid sequence according to SEQ ID NO 8 or 9. Preferably, the Uracil-DNA glycosylase inhibitor comprises an amino acid sequence according to SEQ ID NO 10 or 11.

According to several embodiments, the enzyme comprises the following domain structure, shown in N->C direction:

RuvC-I— Recognition lobe—RuvC-II— deaminase—RuvC-III— PI or

RuvC-I— Recognition lobe—RuvC-II— reverse transcriptase—RuvC-III— PI or

RuvC-I— Recognition lobe—RuvC-II— methyltransferase—RuvC-III— PI or

RuvC-I— Recognition lobe—RuvC-II— transposase—RuvC-III— PI or

RuvC-I— Recognition lobe—RuvC-II— polymerase—RuvC-III— PI or

RuvC-I— Recognition lobe—RuvC-II— nuclease—RuvC-III— PI

with “—” being optional linkers, and optionally
(i) a nuclear localization sequence (NLS) at the C-terminus and/or the N-terminus and/or
(ii) at least one Uracil-DNA glycosylase inhibitor (UGI) domain at the N-terminus.

Preferably, the domain structure is as follows:

NLS—RuvC-I—Recognition lobe—RuvC-II—Deaminase—RuvC-III— PI—NLS

NLS—RuvC-I—Recognition lobe—RuvC-II—Deaminase—RuvC-III— PI—UGI—NLS

NLS—RuvC-I—Recognitionlobe—RuvC-II—Deaminase—RuvC-III— PI—UGI—UGI—NLS or

NL S—RuvC-I—Recogniti on I ob e—RuvC-II—Reverse Transcriptase—RuvC-III— PI—NLS

NLS—RuvC-I—Recognition lobe—RuvC-II—Reverse Transcriptase—RuvC-III— PI—UGI—NLS

NLS—RuvC—I—Recognition lobe—RuvC—II—Reverse Transcriptase—RuvC-III— PI—UGI—UGI—

NLS

According to one embodiment, the enzyme comprises an amino acid sequence according to SEQ ID NOs 12-14, or a sequence having at least 80% sequence identity therewith aet maintaining the targeted deaminase or transcriptase activity.

In some embodiments, the enzyme comprises an amino acid sequence that has >81%, preferably >82%, more preferably >83%, >84%, >85%, >86%, >87%, >88%, >89%, >90%, >91%, >92%, >93%, >94%, >95%, >96%, >97%, >98 or most preferably >99% sequence identity with SEQ ID NOs 12-14.

According to another aspect of the invention, a nucleic acid encoding for the enzyme of the above description is provided. Preferably, said nucleic acid is a DNA or an mRNA.

According to another aspect of the invention, a vector comprising such nucleic acid according is provided.

According to another aspect of the invention, a combination comprising the enzyme or the nucleic acid or the vector of the above description is provided with at least one of
a) a combination of a crRNA and a tracrRNA,
b) a single guide RNA, and/or
c) a pegRNA

As used herein, the term “CRISPR RNA” (crRNAs) relates to a small RNA the sequence of which is complementary or is homologous to the sequence of DNA strand that is to be edited, hence guiding the Cas enzyme to the region of interest.

As used herein, the term “trans-activating crRNA” (tracrRNA) relates to a small trans-encoded RNA that is capable of forming a complex with a CRISPR Cas enzyme. TracrRNA is partially complementary to and base pairs with a crRNA forming an RNA duplex. The combination of tracrRNA and crRNA enables the Cas enzyme to cleave the target DNA in a site specific manner.

As used herein, the term “single guide RNA (sgRNA)” relates to a chimeric RNA molecule that contains the crRNA (targeting sequence) and the tracrRNA (Cas nuclease-recruiting sequence), connected to one another by a short sequence stretch that is optionally palindromic, to form a loop.

As used herein, the term “prime editing guide RNA” relates to chimeric RNA that is used in prime editing, comprising, essentially, a sgRNA plus a further RNA stretch that serves as a template for the reverse transcriptase to synthesize a new DNA sequence.

According to another aspect of the invention, a method for editing a nucleobase and/or reversing a single nucleotide polymorphism within a nucleotide sequence is provided, the method comprising:

(a) contacting said nucleotide sequence with the combination according to the above description, and

(b) converting a first nucleobase of said nucleotide sequence to a second nucleobase, or reversing the single nucleotide polymorphism.

As used herein, the term “reversing a single nucleotide polymorphism” refers to an approach to edit the pathogenic nucleotide in a single nucleotide polymorphism. In such way the wildtype nucleotide is installed.

According to one embodiment, said first nucleobase is adenine or guanine, and said second nucleobase is inosine or uracil.

According to one embodiment, a third nucleobase complementary to said first nucleobase is replaced by a fourth nucleobase complementary to said second nucleobase.

According to one embodiment, the contacting takes place ex vivo/in vitro, or in vivo.

### Examples

While the invention has been illustrated and described in detail in the drawings and foregoing description, such illustration and description are to be considered illustrative or exemplary and not restrictive; the invention is not limited to the disclosed embodiments. Other variations to the disclosed embodiments can be understood and effected by those skilled in the art in practicing the claimed invention, from a study of the drawings, the disclosure, and the appended claims. In the claims, the word “comprising” does not exclude other elements or steps, and the indefinite article “a” or “an” does not exclude a plurality. The mere fact that certain measures are recited in mutually different dependent claims does not indicate that a combination of these measures cannot be used to advantage. Any reference signs in the claims should not be construed as limiting the scope.

All amino acid sequences disclosed herein are shown from N-terminus to C-terminus; all nucleic acid sequences disclosed herein are shown 5′->3′.

Short description of the experiments

Different constructs are rationally engineered and cloned on plasmids. Plasmids encoding these constructs (i.e. deaminases with different linkers) were transfected in HEK293T cells to target endogenous loci on genomic DNA. After 5 days, cells were harvested, their genomic DNA isolated and target loci amplified using PCR. Amplicons were sequenced using an Illumina Miseq sequencer and editing determined using a previously published matlab script.

Methods

General methods and cloning. PCR was performed using Q5 High-Fidelity DNA Polymerase (New England Biolabs). All base editor constructs were assembled using NEBuilder HiFi DNA Assembly (New England Biolabs). Plasmids expressing sgRNAs were cloned using T4 DNA Ligase (New England Biolabs).

Cell culture and high-throughput sequencing. HEK293T cells (ATCC CRL-3216) were cultured in Dulbecco's modified Eagle's medium GlutaMax (Thermo Fisher Scientific), supplemented with 10% (v/v) fetal bovine serum (FBS) and lx penicillin-streptomycin

(Thermo Fisher Scientific) at 37° C. and 5% CO2. Cells were maintained at confluency below 90% and seeded on 96-well cell culture plates (Greiner). 12-16h after seeding, at approximately 70% confluency, cells were transfected using 0.5 μl Lipofectamine 2000 (Thermo Fisher Scientific) and 400 ng base editor plasmid DNA and 10Ong sgRNA plasmids. Cells were incubated for 5 days. Genomic DNA was isolated by adding 10 μl lysis buffer (10 mM Tris-HCl at pH8.0, 2% Triton X and 1 mM EDTA and 25 μg/ml Proteinase K) to 30 μl cell suspension. The lysate was incubated at 60° C. for 60 min, followed by a 95° C. incubation for 10 min. The lysate was diluted with ddH2O to a final volume of 100 μl. 2 μl of the diluted lysate was used for subsequent PCR reactions of 10₁11 using NEBNext High-Fidelity 2x PCR Master Mix. The PCR product was purified using Agencourt AMPure XP beads (Beckman Coulter), and amplified with primers containing sequencing adapters. The products were gel purified and quantified using the Qubit 3.0 fluorometer with the dsDNA HS assay kit (Thermo Fisher Scientific). Samples were sequenced on an Illumina Miseq.

HTS data analysis. Sequencing reads were demultiplexed using Miseq Reporter (Illumina), and analysed using a Matlab as previously described′. Values are shown as n=3 independent biological replicates over different days, with mean±s.d.

Microscopy. HEK293T cells were transfected with 5Ong GFP-expressing plasmids in a 96 well plate and counterstained with Hoechst 33342 and imaged using a Zeiss Apotome. Imaging conditions and intensity scales were matched for all images. Images were analysed using Fiji ImageJ software (v1.51n).

Linker determination and testing. Structural data from SpCas9 (PDB: 5F9R) was used to estimate linker lengths flanking deaminases. Different constructs with combinations of N-and C-terminal linkers were tested, editing efficiencies and activity windows determined by high-throughput sequencing.

FIG. 1A: Lower bar: An adenosine deaminase base editor (ABE) was engineered by integrating a laboratory evolved TadA deaminase into the PI domain (PAM-interacting domain) of a SpCas9 enzyme (called ABEmaxPIl herein, ABEmaxPI2 and ABEmaxPI3 follow a similar concept, but have different linkers flanking the TadA domain).

Upper bar shows then domain structure of a base editor according Gaudelli et al (2017), with the TadA deaminase fused to the N-terminus of the SpCas9 enzyme (called ABEmax herein).

FIG. 1B: ABEmaxPI1, 2 and 3 allowed to extend the editing window PAM-proximally, relative to ABEmax.

FIG. 2A: An adenosine deaminase base editor (ABE) was engineered by replacing the HNH domain of a SpCas9 enzyme by a laboratory evolved TadA deaminase (called HNHx TadA ABE, or, simplified, HNHx ABE, herein)

FIG. 1D: Domain structure of a base editor similar to ABEmax, with the TadA deaminase fused to the N-terminus of the SpCas9 enzyme, yet with the HNH domain replaced by a GGS linker (called ABEmax AHNH herein).

FIG. 1C: Structural data suggest that the HNH nuclease domain (775-908) in SpCas9 likely is a steric hindrance, preventing the deaminase from fully accessing its ssDNA substrate at positions 10 and higher (see arrow). This is critical as the resulting ssDNA is the substrate for deamination. Moreover, while the HNH nuclease domain is essential for cleavage nickase activity, we and others show that catalytically dead ABEs retain similarly high editing efficiencies as the most commonly used nickase ABEs (FIG. 1). We therefore suspected that omission of the HNH domain might improve accessibility and editing at these positions.

FIG. 1E: Transfection of the different constructs in HEK293T cells showed that ABEmax AHNH, lacking the HNH domain, enabled editing at positions 12 and 14, compared to full length ABEmax and dABEmax, albeit at relatively low efficiency. While highest editing rates remained at positions that were also efficiently targeted with full-length ABE constructs (FIG. 1D), the results demonstrate that omission of the HNH domain expands the editing window.

FIG. 2B: Replacing the HNH domain with the adenine deaminase allows to shift the editing window PAM-proximally. Before incorporating a deaminase domain in place of the HNH domain in SpCas9, a superfolder (sf)GFP was inserted to assess the viability of this approach.

FIG. 2C: Notably, AHNH-sfGFP fusions were green fluorescent and localized to the nucleus.

FIG. 2D: In a next step, we tested engineered HNHx-ABE variants by incorporating the evolved deaminase domain from ABEmax (See FIG. 2A for schematic) with different protein linkers into SpCas9 lacking the HNH domain.

Using high throughput sequencing (HTS), editing efficiencies of 20 different constructs with different linker combinations were compared. The most promising candidate, containing a GGS-linker to join SpCas9 S793 and the TadA N-terminus and a SGG-linker to join the TadA C-terminus and SpCas9 R919, demonstrated a clear shift in the editing window towards the PAM domain, with up to 13% editing (see arrow).

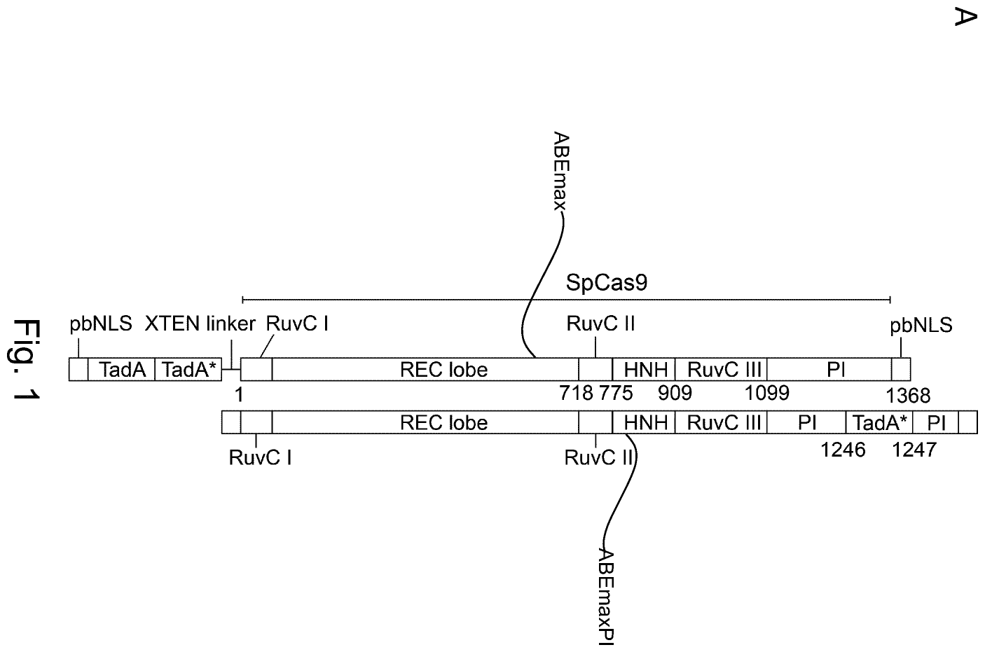

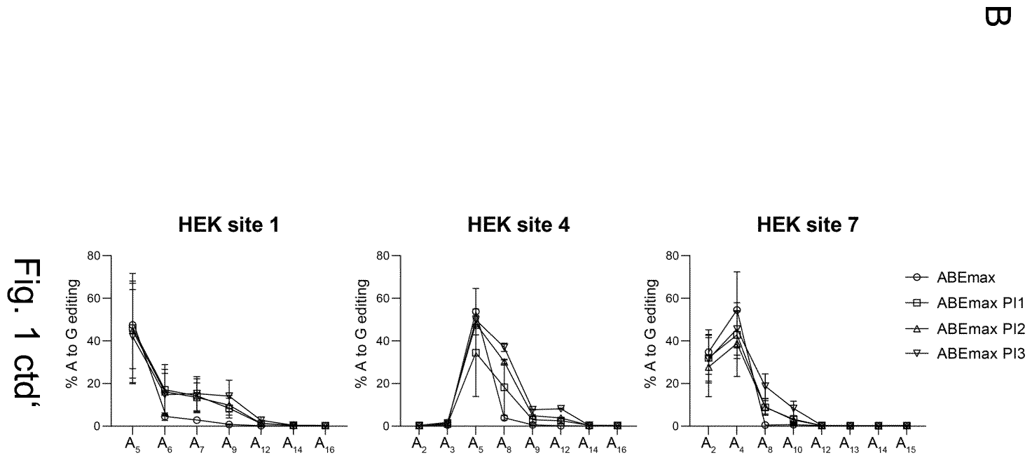

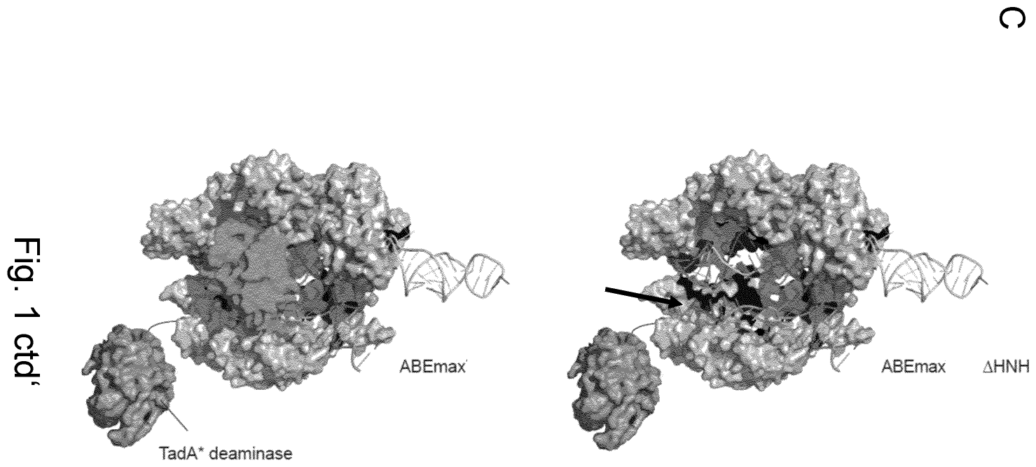

FIG. 3: Testing this variant on additional endogenous loci further confirmed this observation.

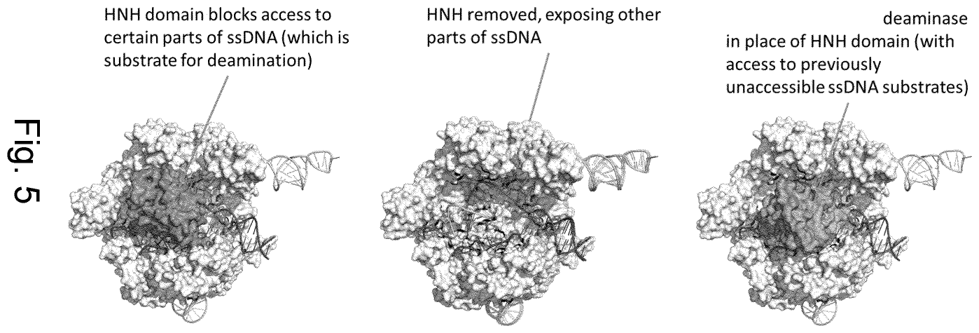

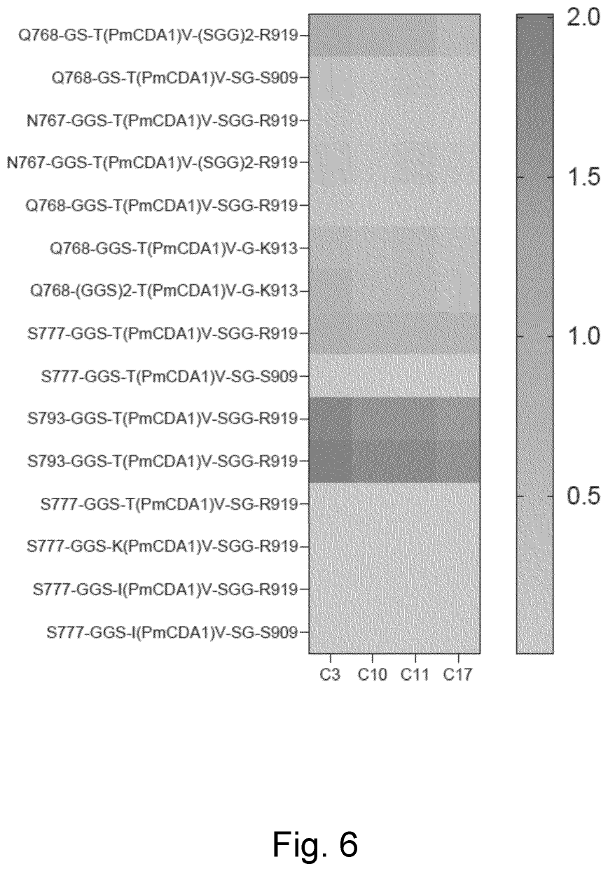

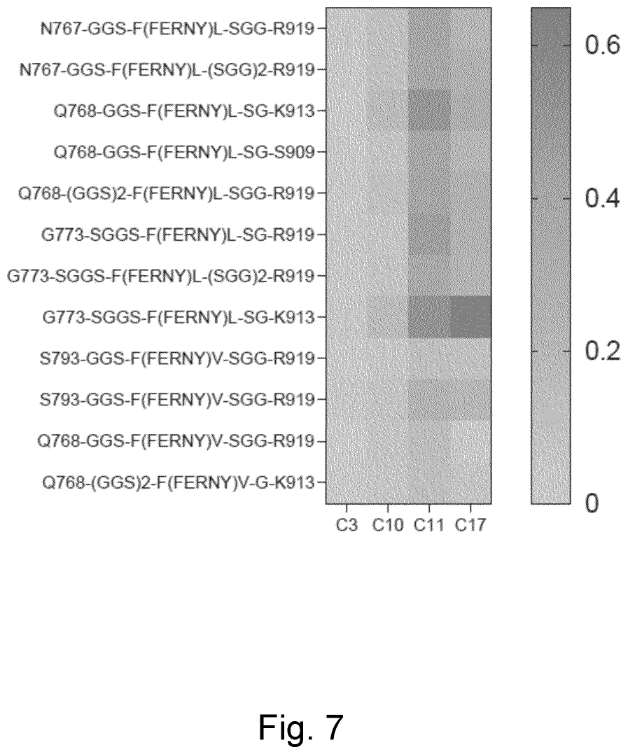

Using the same approach, we have also inserted cytosine deaminase domains, including PmCDA1, rAPOBEC1, and FERNY, which is an evolved APOBEC variant in place of the HNH domain in SpCas9. While the editing window was also shifted, efficiencies of these HNH-cytidine base editor (CBE) variants were substantially lower compared to dHNH-ABE variants (FIG. 6, 7).

Taken together, it has been demonstrated that the editing window of Base Editors (BE) can be shifted by replacing the HNH domain with a deaminase domain, extending their targeting scope. Although current. Replacement of the HNH domain with a TadA further reduces the size of typical BEs from 5.2 kb to 4.3 kb, potentially enabling them to be packaged on Adeno-associated virus (AAVs). Cas9 HNH nuclease domains can also be replaced with other genome editing enzymes that act on single-stranded DNA, hence yielding similar advantages.

### REFERENCES

Thuronyi et al., Nat Biotechnol. 2019 September;37(9):1070-1079 Anzalone et al., Nature (2019) doi:10.1038/s41586-019-1711-4 Kim et al., Nat Biotechnol 35, 371-376 (2017) Gaudelli et al., Nature 559, E8 (2018) doi:10.1038/s41586-018-0070-x Komor et al., Nature 533, 420-424 (2016) doi:10.1038/nature17946

Rees and Liu, Base editing: precision chemistry on the genome and transcriptome of living cells. Nat Rev Genet. 2018 December; 19(12): 770-788 Gaudelli et al. Programmable base editing of A*T to G*C in genomic DNA without DNA cleavage. Nature 551,464-471,10.1038/nature24644 (2017)

Sequences

The following sequences form part of the disclosure of the present application. A WIPO ST 25 compatible electronic sequence listing is provided with this application, too. For the avoidance of doubt, if discrepancies exist between the sequences in the following table and the electronic sequence listing, the sequences in this table shall be deemed to be the correct ones.

Underlined: Nuclear Localization Sequence (NLS) Double underlined: deaminase (tadA, Apobecl, pmCDA1 or ferny) Italics: Linker Bold: uracil DNA glycosylase inhibitor (UGI)

Note that with regard to optional mutations, the N-terminal M residue of some deaminases may be not be included into the counting. When introduced into the Cas9 backbone, the M residue is removed.

| | | Uni- | | Optional |
|---|---|---|---|---|
| No | Type | prot | Sequence | Mutations |
| 1 | SpCas9 | Q99ZW2 | MDKKYSIGLDIGTNSVGWAVITDEYKVPSKKFKVLGNTDRHSIKKNLIGALLFDSGETAEATR | D10 |
| | | | LKRTARRRYTRRKNRICYLQEIFSNEMAKVDDSFFHRLEESFLVEEDKKHERHPIFGNIVDEVA | |
| | | | YHEKYPTIYHLRKKLVDSTDKADLRLIYLALAHMIKFRGHFLIEGDLNPDNSDVDKLFIQLVQT | |
| | | | YNQLFEENPINASGVDAKAILSARLSKSRRLENLIAQLPGEKKNGLFGNLIALSLGLTPNFKSNF | |
| | | | DLAEDAKLQLSKDTYDDDLDNLLAQIGDQYADLFLAAKNLSDAILLSDILRVNTEITKAPLSAS | |
| | | | MIKRYDEHHQDLTLLKALVRQQLPEKYKEIFFDQSKNGYAGYIDGGASQEEFYKFIKPILEKM | |
| | | | DGTEELLVKLNREDLLRKQRTFDNGSIPHQIHLGELHAILRRQEDFYPFLKDNREKIEKILTFRI | |
| | | | PYYVGPLARGNSRFAWMTRKSEETITPWNFEEVVDKGASAQSFIERMTNFDKNLPNEKVLP | |
| | | | KHSLLYEYFTVYNELTKVKYVTEGMRKPAFLSGEQKKAIVDLLFKTNRKVTVKQLKEDYFKKIE | |
| | | | CFDSVEISGVEDRFNASLGTYHDLLKIIKDKDFLDNEENEDILEDIVLTLTLFEDREMIEERLKTY | |
| | | | AHLFDDKVMKQLKRRRYTGWGRLSRKLINGIRDKQSGKTILDFLKSDGFANRNFMQLIHDD | |
| | | | SLTFKEDIQKAQVSGQGDSLHEHIANLAGSPAIKKGILQTVKVVDELVKVMGRHKPENIVIE | |
| | | | MARENQTTQKGQKNSRERMKRIEEGIKELGSQILKEHPVENTQLQNEKLYLYYLQNGRDM | |
| | | | YVDQELDINRLSDYDVDHIVPQSFLKDDSIDNKVLTRSDKNRGKSDNVPSEEVVKKMKNYW | |
| | | | RQLLNAKLITQRKFDNLTKAERGGLSELDKAGFIKRQLVETRQITKHVAQILDSRMNTKYDEN | |
| | | | DKLIREVKVITLKSKLVSDFRKDFQFYKVREINNYHHAHDAYLNAVVGTALIKKYPKLESEFVY | |
| | | | GDYKVYDVRKMIAKSEQEIGKATAKYFFYSNIMNFFKTEITLANGEIRKRPLIETNGETGEIVW | |
| | | | DKGRDFATVRKVLSMPQVNIVKKTEVQTGGFSKESILPKRNSDKLIARKKDWDPKKYGGFD | |
| | | | SPTVAYSVLVVAKVEKGKSKKLKSVKELLGITIMERSSFEKNPIDFLEAKGYKEVKKDLIIKLPKY | |
| | | | SLFELENGRKRMLASAGELQKGNELALPSKYVNFLYLASHYEKLKGSPEDNEQKQLFVEQHK | |
| | | | HYLDEIIEQISEFSKRVILADANLDKVLSAYNKHRDKPIREQAENIIHLFTLTNLGAPAAFKYFDT | |
| | | | TIDRKRYTSTKEVLDATLIHQSITGLYETRIDLSQLGGD | |
| 2 | SaCas9 | J7RUA5 | MKRNYILGLDIGITSVGYGIIDYETRDVIDAGVRLFKEANVENNEGRRSKRGARRLKRRRRHR | D10 |
| | | | IQRVKKLLFDYNLLTDHSELSGINPYEARVKGLSQKLSEEEFSAALLHLAKRRGVHNVNEVEE | |
| | | | DTGNELSTKEQISRNSKALEEKYVAELQLERLKKDGEVRGSINRFKTSDYVKEAKQLLKVQKA | |
| | | | YHQLDQSFIDTYIDLLETRRTYYEGPGEGSPFGWKDIKEWYEMLMGHCTYFPEELRSVKYAY | |
| | | | NADLYNALNDLNNLVITRDENEKLEYYEKFQIIENVFKQKKKPTLKQIAKEILVNEEDIKGYRVT | |
| | | | STGKPEFTNLKVYHDIKDITARKEIIENAELLDQIAKILTIYQSSEDIQEELTNLNSELTQEEIEQIS | |
| | | | NLKGYTGTHNLSLKAINLILDELWHTNDNQIAIFNRLKLVPKKVDLSQQKEIPTTLVDDFILSP | |
| | | | VVKRSFIQSIKVINAIIKKYGLPNDIIIELAREKNSKDAQKMINEMQKRNRQTNERIEEIIRTTGK | |
| | | | ENAKYLIEKIKLHDMQEGKCLYSLEAIPLEDLLNNPFNYEVDHIIPRSVSFDNSFNNKVLVKQE | |
| | | | ENSKKGNRTPFQYLSSSDSKISYETFKKHILNLAKGKGRISKTKKEYLLEERDINRFSVQKDFIN | |
| | | | RNLVDTRYATRGLMNLLRSYFRVNNLDVKVKSINGGFTSFLRRKWKFKKERNKGYKHHAED | |
| | | | ALIIANADFIFKEWKKLDKAKKVMENQMFEEKQAESMPEIETEQEYKEIFITPHQIKHIKDFK | |
| | | | DYKYSHRVDKKPNRELINDTLYSTRKDDKGNTLIVNNLNGLYDKDNDKLKKLINKSPEKLLMY | |
| | | | HHDPQTYQKLKLIMEQYGDEKNPLYKYYEETGNYLTKYSKKDNGPVIKKIKYYGNKLNAHLDI | |
| | | | TDDYPNSRNKVVKLSLKPYRFDVYLDNGVYKFVTVKNLDVIKKENYYEVNSKCYEEAKKLKKI | |
| | | | SNQAEFIASFYNNDLIKINGELYRVIGVNNDLLNRIEVNMIDITYREYLENMNDKRPPRIIKTIA | |
| | | | SKTQSIKKYSTDILGNLYEVKSKKHPQIIKKG | |
| 3 | StCas9 | G3ECR1 | MLFNKCIIISINLDFSNKEKCMTKPYSIGLDIGTNSVGWAVITDNYKVPSKKMKVLGNTSKKYI | D14, D31 |
| | | | KKNLLGVLLFDSGITAEGRRLKRTARRRYTRRRNRILYLQEIFSTEMATLDDAFFQRLDDSFLV | |
| | | | PDDKRDSKYPIFGNLVEEKVYHDEFPTIYHLRKYLADSTKKADLRLVYLALAHMIKYRGHFLIE | |
| | | | GEFNSKNNDIQKNFQDFLDTYNAIFESDLSLENSKQLEEIVKDKISKLEKKDRILKLFPGEKNSG | |
| | | | IFSEFLKLIVGNQADFRKCFNLDEKASLHFSKESYDEDLETLLGYIGDDYSDVFLKAKKLYDAILL | |
| | | | SGFLTVTDNETEAPLSSAMIKRYNEHKEDLALLKEYIRNISLKTYNEVFKDDTKNGYAGYIDGK | |
| | | | TNQEDFYVYLKNLLAEFEGADYFLEKIDREDFLRKQRTFDNGSIPYQIHLQEMRAILDKQAKF | |
| | | | YPFLAKNKERIEKILTFRIPYYVGPLARGNSDFAWSIRKRNEKITPWNFEDVIDKESSAEAFINR | |
| | | | MTSFDLYLPEEKVLPKHSLLYETFNVYNELTKVRFIAESMRDYQFLDSKQKKDIVRLYFKDKRK | |
| | | | VTDKDIIEYLHAIYGYDGIELKGIEKQFNSSLSTYHDLLNIINDKEFLDDSSNEAIIEEIIHTLTIFED | |
| | | | REMIKQRLSKFENIFDKSVLKKLSRRHYTGWGKLSAKLINGIRDEKSGNTILDYLIDDGISNRN | |
| | | | FMQLIHDDALSFKKKIQKAQIIGDEDKGNIKEVVKSLPGSPAIKKGILQSIKIVDELVKVMGGR | |
| | | | KPESIVVEMARENQYTNQGKSNSQQRLKRLEKSLKELGSKILKENIPAKLSKIDNNALQNDRL | |
| | | | YLYYLQNGKDMYTGDDLDIDRLSNYDIDHIIPQAFLKDNSIDNKVLVSSASNRGKSDDFPSLE | |
| | | | VVKKRKTFWYQLLKSKLISQRKFDNLTKAERGGLLPEDKAGFIQRQLVETRQITKHVARLLDE | |
| | | | KFNNKKDENNRAVRTVKIITLKSTLVSQFRKDFELYKVREINDFHHAHDAYLNAVIASALLKKY | |
| | | | PKLEPEFVYGDYPKYNSFRERKSATEKVYFYSNIMNIFKKSISLADGRVIERPLIEVNEETGESV | |
| | | | WNKESDLATVRRVLSYPQVNVVKKVEEQNHGLDRGKPKGLFNANLSSKPKPNSNENLVGA | |
| | | | KEYLDPKKYGGYAGISNSFAVLVKGTIEKGAKKKITNVLEFQGISILDRINYRKDKLNFLLEKGY | |
| | | | KDIELIIELPKYSLFELSDGSRRMLASILSTNNKRGEIHKGNQIFLSQKFVKLLYHAKRISNTINE | |
| | | | NHRKYVENHKKEFEELFYYILEFNENYVGAKKNGKLLNSAFQSWQNHSIDELCSSFIGPTGSE | |
| | | | RKGLFELTSRGSAADFEFLGVKIPRYRDYTPSSLLKDATLIHQSVTGLYETRIDLAKLGEG | |
| 4 | tadA | P68398 | MSEVEFSHEYWMRHALTLAKRAWDEREVPVGAVLVHNNRVIGEGWNRPIGRHDPTAHA | A106V, D108N, |
| | | | EIMALRQGGLVMQNYRLIDATLYVTLEPCVMCAGAMIHSRIGRVVFGARDAKTGAAGSLM | D147Y, L84F, |
| | | | DVLHHPGMNHRVEITEGILADECAALLSDFFRMRRQEIKAQKKAQSSTD | H123Y, I157F, |
| | | | | and/or E155V |
| 5 | Apobec1 | P41238 | MTSEKGPSTGDPTLRRRIEPWEFDVFYDPRELRKEACLLYEIKWGMSRKIWRSSGKNTTNH | |
| | | | VEVNFIKKFTSERDFHPSMSCSITWFLSWSPCWECSQAIREFLSRHPGVTLVIYVARLFWHM | |
| | | | DQQNRQGLRDLVNSGVTIQIMRASEYYHCWRNFVNYPPGDEAHWPQYPPLWMMLYAL | |
| | | | ELHCIILSLPPCLKISRRWQNHLTFFRLHLQNCHYQTIPPHILLATGLIHPSVAWR | |
| 6 | pmCDA1 | A5H718 | MTDAEYVRIHEKLDIYTFKKQFFNNKKSVSHRCYVLFELKRRGERRACFWGYAVNKPQSGTE | F22S, A123V, |
| | | | RGIHAEIFSIRKVEEYLRDNPGQFTINWYSSWSPCADCAEKILEWYNQELRGNGHTLKIWAC | and/or I195F |
| | | | KLYYEKNARNQIGLWNLRDNGVGLNVMVSEHYQCCRKIFIQSSHNQLNENRWLEKTLKRA | |
| | | | EKRRSELSIMIQVKILHTTKSPAV | |
| 7 | ferny | | FERNYDPRELRKETYLLYEIKWGKSGKLWRHWCQNNRTQHAEVYFLENIFNARRFNPSTHC | |
| | | | SITWYLSWSPCAECSQKIVDFLKEHPNVNLEIYVARLYYPENERNRQGLRDLVNSGVTIRIMD | |
| | | | LPDYNYCWKTFVSDQGGDEDYWPGHFAPWIKQYSLKL | |
| 8 | NLS | | MKRTADGSEFESPKKKRKV | |
| 9 | NLS | | KRTADGSEFESPKKKRKV | |
| 10 | UGI | P14739 | TNLSDHEKETGKQLVIQESILMLPEEVEEVIGNKPESDILVHTAYDESTDENVMLLTSDAPE | |
| | | | YKPWALVIQDSNGENKIKML | |
| 11 | UGI | | TNLSDIIEKETGKQLVIQESILMLPEEVEEVIGNKPESDILVHTAYDESTDENVMLLTSDAPE | |
| | | | YKPWALVIQDSNGENKIKMLSGGSGGSGGSTNLSDIIEKETGKQLVIQESILMLPEEVEEVI | |
| | | | GNKPESDILVHTAYDESTDENVMLLTSDAPEYKPWALVIQDSNGENKIKML | |
| 12 | HNHx_ | | MKRTADGSEFESPKKKRKVGSDKKYSIGLAIGTNSVGWAVITDEYKVPSKKFKVLGNTDRHSI | D10A (counted |
| | TadA_ | | KKNLIGALLFDSGETAEATRLKRTARRRYTRRKNRICYLQEIFSNEMAKVDDSFFHRLEESFLV | without NLS) |
| | ABE | | EEDKKHERHPIFGNIVDEVAYHEKYPTIYHLRKKLVDSTDKADLRLIYLALAHMIKFRGHFLIEG | A106V/ |
| | | | DLNPDNSDVDKLFIQLVQTYNQLFEENPINASGVDAKAILSARLSKSRRLENLIAQLPGEKKN | D108ND147Y/ |
| | | | GLFGNLIALSLGLTPNFKSNFDLAEDAKLQLSKDTYDDDLDNLLAQIGDQYADLFLAAKNLSD | E155V (counted |
| | | | AILLSDILRVNTEITKAPLSASMIKRYDEHHQDLTLLKALVRQQLPEKYKEIFFDQSKNGYAGYI | without NLS) |
| | | | DGGASQEEFYKFIKPILEKMDGTEELLVKLNREDLLRKQRTFDNGSIPHQIHLGELHAILRRQE | |
| | | | DFYPFLKDNREKIEKILTFRIPYYVGPLARGNSRFAWMTRKSEETITPWNFEEVVDKGASAQS | |
| | | | FIERMTNFDKNLPNEKVLPKHSLLYEYFTVYNELTKVKYVTEGMRKPAFLSGEQKKAIVDLLF | |
| | | | KTNRKVTVKQLKEDYFKKIECFDSVEISGVEDRFNASLGTYHDLLKIIKDKDFLDNEENEDILED | |
| | | | IVLTLTLFEDREMIEERLKTYAHLFDDKVMKQLKRRRYTGWGRLSRKLINGIRDKQSGKTILDF | |
| | | | LKSDGFANRNFMQLIHDDSLTFKEDIQKAQVSGQGDSLHEHIANLAGSPAIKKGILQTVKVV | |
| | | | DELVKVMGRHKPENIVIEMARENQTTQKGQKNSRERMKRIEEGIKELGSGGSSEVEFSHEY | |
| | | | WMRHALTLAKRARDEREVPVGAVLVLNNRVIGEGWNRAIGLHDPTAHAEIMALRQGGLV | |
| | | | MQNYRLIDATLYVTFEPCVMCAGAMIHSRIGRVVFGVRNAKTGAAGSLMDVLHYPGMNH | |
| | | | RVEITEGILADECAALLCYFFRMPRQVFNAQKKAQSSTDSGGRQLVETRQITKHVAQILDSR | |
| | | | MNTKYDENDKLIREVKVITLKSKLVSDFRKDFQFYKVREINNYHHAHDAYLNAVVGTALIKKY | |
| | | | PKLESEFVYGDYKVYDVRKMIAKSEQEIGKATAKYFFYSNIMNFFKTEITLANGEIRKRPLIETN | |
| | | | GETGEIVWDKGRDFATVRKVLSMPQVNIVKKTEVQTGGFSKESILPKRNSDKLIARKKDWD | |
| | | | PKKYGGFDSPTVAYSVLVVAKVEKGKSKKLKSVKELLGITIMERSSFEKNPIDFLEAKGYKEVK | |
| | | | KDLIIKLPKYSLFELENGRKRMLASAGELQKGNELALPSKYVNFLYLASHYEKLKGSPEDNEQK | |
| | | | QLFVEQHKHYLDEIIEQISEFSKRVILADANLDKVLSAYNKHRDKPIREQAENIIHLFTLTNLGA | |
| | | | PAAFKYFDTTIDRKRYTSTKEVLDATLIHQSITGLYETRIDLSQLGGDSGGSKRTADGSEFESPK | |
| | | | KKRKV | |
| 13 | HNHx_ | | MKRTADGSEFESPKKKRKVGSDKKYSIGLAIGTNSVGWAVITDEYKVPSKKFKVLGNTDRHSI | D10 (counted |
| | FERNY_ | | KKNLIGALLFDSGETAEATRLKRTARRRYTRRKNRICYLQEIFSNEMAKVDDSFFHRLEESFLV | without NLS) |
| | ABE | | EEDKKHERHPIFGNIVDEVAYHEKYPTIYHLRKKLVDSTDKADLRLIYLALAHMIKFRGHFLIEG | |
| | | | DLNPDNSDVDKLFIQLVQTYNQLFEENPINASGVDAKAILSARLSKSRRLENLIAQLPGEKKN | |
| | | | GLFGNLIALSLGLTPNFKSNFDLAEDAKLQLSKDTYDDDLDNLLAQIGDQYADLFLAAKNLSD | |
| | | | AILLSDILRVNTEITKAPLSASMIKRYDEHHQDLTLLKALVRQQLPEKYKEIFFDQSKNGYAGYI | |
| | | | DGGASQEEFYKFIKPILEKMDGTEELLVKLNREDLLRKQRTFDNGSIPHQIHLGELHAILRRQE | |
| | | | DFYPFLKDNREKIEKILTFRIPYYVGPLARGNSRFAWMTRKSEETITPWNFEEVVDKGASAQS | |
| | | | FIERMTNFDKNLPNEKVLPKHSLLYEYFTVYNELTKVKYVTEGMRKPAFLSGEQKKAIVDLLF | |
| | | | KTNRKVTVKQLKEDYFKKIECFDSVEISGVEDRFNASLGTYHDLLKIIKDKDFLDNEENEDILED | |
| | | | IVLTLTLFEDREMIEERLKTYAHLFDDKVMKQLKRRRYTGWGRLSRKLINGIRDKQSGKTILDF | |
| | | | LKSDGFANRNFMQLIHDDSLTFKEDIQKAQVSGQGDSLHEHIANLAGSPAIKKGILQTVKVV | |
| | | | DELVKVMGRHKPENIVIEMARENQTTQKGSGGSGSFERNYDPRELRKETYLLYEIKWGKSG | |
| | | | KLWRHWCQNNRTQHAEVYFLENIFNARRFNPSTHCSITWYLSWSPCAECSQKIVDFLKEHP | |
| | | | NVNLEIYVARLYYPENERNRQGLRDLVNSGVTIRIMDLPDYNYCWKTFVSDQGGDEDYWP | |
| | | | GHFAPWIKQYSLKLSGKAGFIKRQLVETRQITKHVAQILDSRMNTKYDENDKLIREVKVITLK | |
| | | | SKLVSDFRKDFQFYKVREINNYHHAHDAYLNAVVGTALIKKYPKLESEFVYGDYKVYDVRKM | |
| | | | IAKSEQEIGKATAKYFFYSNIMNFFKTEITLANGEIRKRPLIETNGETGEIVWDKGRDFATVRK | |
| | | | VLSMPQVNIVKKTEVQTGGFSKESILPKRNSDKLIARKKDWDPKKYGGFDSPTVAYSVLVVA | |
| | | | KVEKGKSKKLKSVKELLGITIMERSSFEKNPIDFLEAKGYKEVKKDLIIKLPKYSLFELENGRKRM | |
| | | | LASAGELQKGNELALPSKYVNFLYLASHYEKLKGSPEDNEQKQLFVEQHKHYLDEIIEQISEFS | |
| | | | KRVILADANLDKVLSAYNKHRDKPIREQAENIIHLFTLTNLGAPAAFKYFDTTIDRKRYTSTKE | |
| | | | VLDATLIHQSITGLYETRIDLSQLGGDSGGSGGSGGSTNLSDIIEKETGKQLVIQESILMLPEE | |
| | | | VEEVIGNKPESDILVHTAYDESTDENVMLLTSDAPEYKPWALVIQDSNGENKIKMLSGGS | |
| | | | GGSGGSTNLSDIIEKETGKQLVIQESILMLPEEVEEVIGNKPESDILVHTAYDESTDENVMLL | |
| | | | TSDAPEYKPWALVIQDSNGENKIKMLSGGSKRTADGSEFESPKKKRKV | |
| 14 | HNHx_ | | MKRTADGSEFESPKKKRKVGSDKKYSIGLAIGTNSVGWAVITDEYKVPSKKFKVLGNTDRHSI | D10 (counted |
| | pmCDA1_ | | KKNLIGALLFDSGETAEATRLKRTARRRYTRRKNRICYLQEIFSNEMAKVDDSFFHRLEESFLV | without NLS) |
| | ABE | | EEDKKHERHPIFGNIVDEVAYHEKYPTIYHLRKKLVDSTDKADLRLIYLALAHMIKFRGHFLIEG | F22S, A123V, |
| | | | DLNPDNSDVDKLFIQLVQTYNQLFEENPINASGVDAKAILSARLSKSRRLENLIAQLPGEKKN | I195F (counted |
| | | | GLFGNLIALSLGLTPNFKSNFDLAEDAKLQLSKDTYDDDLDNLLAQIGDQYADLFLAAKNLSD | without NLS) |
| | | | AILLSDILRVNTEITKAPLSASMIKRYDEHHQDLTLLKALVRQQLPEKYKEIFFDQSKNGYAGYI | |
| | | | DGGASQEEFYKFIKPILEKMDGTEELLVKLNREDLLRKQRTFDNGSIPHQIHLGELHAILRRQE | |
| | | | DFYPFLKDNREKIEKILTFRIPYYVGPLARGNSRFAWMTRKSEETITPWNFEEVVDKGASAQS | |
| | | | FIERMTNFDKNLPNEKVLPKHSLLYEYFTVYNELTKVKYVTEGMRKPAFLSGEQKKAIVDLLF | |
| | | | KTNRKVTVKQLKEDYFKKIECFDSVEISGVEDRFNASLGTYHDLLKIIKDKDFLDNEENEDILED | |
| | | | IVLTLTLFEDREMIEERLKTYAHLFDDKVMKQLKRRRYTGWGRLSRKLINGIRDKQSGKTILDF | |
| | | | LKSDGFANRNFMQLIHDDSLTFKEDIQKAQVSGQGDSLHEHIANLAGSPAIKKGILQTVKVV | |
| | | | DELVKVMGRHKPENIVIEMARENQTTQKGQKNSRERMKRIEEGIKELGSTDAEYVRIHEKLD | |
| | | | IYTFKKQFSNNKKSVSHRCYVLFELKRRGERRACFWGYAVNKPQSGTERGIHAEIFSIRKVEE | |
| | | | YLRDNPGQFTINWYSSWSPCADCAEKILEWYNQELRGNGHTLKIWVCKLYYEKNARNQIGL | |
| | | | WNLRDNGVGLNVMVSEHYQCCRKIFIQSSHNQLNENRWLEKTLKRAEKRRSELSIMFQVKI | |
| | | | LHTTKSPAVSGGSGGRQLVETRQITKHVAQILDSRMNTKYDENDKLIREVKVITLKSKLVSDF | |
| | | | RKDFQFYKVREINNYHHAHDAYLNAVVGTALIKKYPKLESEFVYGDYKVYDVRKMIAKSEQEI | |
| | | | GKATAKYFFYSNIMNFFKTEITLANGEIRKRPLIETNGETGEIVWDKGRDFATVRKVLSMPQV | |
| | | | NIVKKTEVQTGGFSKESILPKRNSDKLIARKKDWDPKKYGGFDSPTVAYSVLVVAKVEKGKSK | |
| | | | KLKSVKELLGITIMERSSFEKNPIDFLEAKGYKEVKKDLIIKLPKYSLFELENGRKRMLASAGELQ | |
| | | | KGNELALPSKYVNFLYLASHYEKLKGSPEDNEQKQLFVEQHKHYLDEIIEQISEFSKRVILADA | |
| | | | NLDKVLSAYNKHRDKPIREQAENIIHLFTLTNLGAPAAFKYFDTTIDRKRYTSTKEVLDATLIH | |
| | | | QSITGLYETRIDLSQLGGDSGGSGGSGGSTNLSDIIEKETGKQLVIQESILMLPEEVEEVIGNK | |
| | | | PESDILVHTAYDESTDENVMLLTSDAPEYKPWALVIQDSNGENKIKMLSGGSGGSGGSTN | |
| | | | LSDIIEKETGKQLVIQESILMLPEEVEEVIGNKPESDILVHTAYDESTDENVMLLTSDAPEYK | |
| | | | PWALVIQDSNGENKIKMLSGGSKRTADGSEFESPKKKRKV | |
| 15 | M-MLV RT | | TLNIEDEYRLHETSKEPDVSLGSTWLSDFPQAWAETGGMGLAVRQAPLIIPLKATSTPVSIKQ | D200N, T306K, |
| | | | YPMSQEARLGIKPHIQRLLDQGILVPCQSPWNTPLLPVKKPGTNDYRPVQDLREVNKRVEDI | W313F, T330P, |
| | | | HPTVPNPYNLLSGLPPSHQWYTVLDLKDAFFCLRLHPTSQPLFAFEWRDPEMGISGQLTWT | L603W |
| | | | RLPQGFKNSPTLFDEALHRDLADFRIQHPDLILLQYVDDLLLAATSELDCQQGTRALLQTLGN | |
| | | | LGYRASAKKAQICQKQVKYLGYLLKEGQRWLTEARKETVMGQPTPKTPRQLREFLGTAGFC | |
| | | | RLWIPGFAEMAAPLYPLTKTGTLFNWGPDQQKAYQEIKQALLTAPALGLPDLTKPFELFVDE | |
| | | | KQGYAKGVLTQKLGPWRRPVAYLSKKLDPVAAGWPPCLRMVAAIAVLTKDAGKLTMGQP | |
| | | | LVILAPHAVEALVKQPPDRWLSNARMTHYQALLLDTDRVQFGPVVALNPATLLPLPEEGLQ | |
| | | | HNCLDILAEAHGTRPDLTDQPLPDADHTWYTDGSSLLQEGQRKAGAAVTTETEVIWAKALP | |
| | | | AGTSAQRAELIALTQALKMAEGKKLNVYTDSRYAFATAHIHGEIYRRRGLLTSEGKEIKNKDEI | |
| | | | LALLKALFLPKRLSIIHCPGHQKGHSAEARGNRMADQAARKAAITETPDTSTLLIENSSP | |
| 16 | CjCas9 | Q0P897 | MARILAFDIGISSIGWAFSENDELKDCGVRIFTKVENPKTGESLALPRRLARSARKRLARRKAR | D80 |
| | | | LNHLKHLIANEFKLNYEDYQSFDESLAKAYKGSLISPYELRFRALNELLSKQDFARVILHIAKRR | |
| | | | GYDDIKNSDDKEKGAILKAIKQNEEKLANYQSVGEYLYKEYFQKFKENSKEFTNVRNKKESYE | |
| | | | RCIAQSFLKDELKLIFKKQREFGFSFSKKFEEEVLSVAFYKRALKDFSHLVGNCSFFTDEKRAPK | |
| | | | NSPLAFMFVALTRIINLLNNLKNTEGILYTKDDLNALLNEVLKNGTLTYKQTKKLLGLSDDYEF | |
| | | | KGEKGTYFIEFKKYKEFIKALGEHNLSQDDLNEIAKDITLIKDEIKLKKALAKYDLNQNQIDSLSK | |
| | | | LEFKDHLNISFKALKLVTPLMLEGKKYDEACNELNLKVAINEDKKDFLPAFNETYYKDEVTNP | |
| | | | VVLRAIKEYRKVLNALLKKYGKVHKINIELAREVGKNHSQRAKIEKEQNENYKAKKDAELECE | |
| | | | KLGLKINSKNILKLRLFKEQKEFCAYSGEKIKISDLQDEKMLEIDHIYPYSRSFDDSYMNKVLVF | |
| | | | TKQNQEKLNQTPFEAFGNDSAKWQKIEVLAKNLPTKKQKRILDKNYKDKEQKNFKDRNLN | |
| | | | DTRYIARLVLNYTKDYLDFLPLSDDENTKLNDTQKGSKVHVEAKSGMLTSALRHTWGFSAK | |
| | | | DRNNHLHHAIDAVIIAYANNSIVKAFSDFKKEQESNSAELYAKKISELDYKNKRKFFEPFSGFR | |
| | | | QKVLDKIDEIFVSKPERKKPSGALHEETFRKEEEFYQSYGGKEGVLKALELGKIRKVNGKIVKN | |
| | | | GDMFRVDIFKHKKTNKFYAVPIYTMDFALKVLPNKAVARSKKGEIKDWILMDENYEFCFSLY | |
| | | | KDSLILIQTKDMQEPEFVYYNAFTSSTVSLIVSKHDNKFETLSKNQKILFKNANEKEVIAKSIGI | |
| | | | QNLKVFEKYIVSALGEVTKAEFRQREDFKK | |
| 17 | NmeCas9 | A1IQ68 | MAAFKPNPINYILGLDIGIASVGWAMVEIDEDENPICLIDLGVRVFERAEVPKTGDSLAMAR | D16A |
| | | | RLARSVRRLTRRRAHRLLRARRLLKREGVLQAADFDENGLIKSLPNTPWQLRAAALDRKLTP | |
| | | | LEWSAVLLHLIKHRGYLSQRKNEGETADKELGALLKGVADNAHALQTGDFRTPAELALNKFE | |
| | | | KESGHIRNQRGDYSHTFSRKDLQAELILLFEKQKEFGNPHVSGGLKEGIETLLMTQRPALSGD | |
| | | | AVQKMLGHCTFEPAEPKAAKNTYTAERFIWLTKLNNLRILEQGSERPLTDTERATLMDEPYR | |
| | | | KSKLTYAQARKLLGLEDTAFFKGLRYGKDNAEASTLMEMKAYHAISRALEKEGLKDKKSPLNL | |
| | | | SPELQDEIGTAFSLFKTDEDITGRLKDRIQPEILEALLKHISFDKFVQISLKALRRIVPLMEQGKR | |
| | | | YDEACAEIYGDHYGKKNTEEKIYLPPIPADEIRNPVVLRALSQARKVINGVVRRYGSPARIHIE | |
| | | | TAREVGKSFKDRKEIEKRQEENRKDREKAAAKFREYFPNFVGEPKSKDILKLRLYEQQHGKCL | |
| | | | YSGKEINLGRLNEKGYVEIDHALPFSRTWDDSFNNKVLVLGSENQNKGNQTPYEYFNGKDN | |
| | | | SREWQEFKARVETSRFPRSKKQRILLQKFDEDGFKERNLNDTRYVNRFLCQFVADRMRLTG | |
| | | | KGKKRVFASNGQITNLLRGFWGLRKVRAENDRHHALDAVVVACSTVAMQQKITRFVRYKE | |
| | | | MNAFDGKTIDKETGEVLHQKTHFPQPWEFFAQEVMIRVFGKPDGKPEFEEADTPEKLRTLL | |
| | | | AEKLSSRPEAVHEYVTPLFVSRAPNRKMSGQGHMETVKSAKRLDEGVSVLRVPLTQLKLKDL | |
| | | | EKMVNREREPKLYEALKARLEAHKDDPAKAFAEPFYKYDKAGNRTQQVKAVRVEQVQKTG | |
| | | | VWVRNHNGIADNATMVRVDVFEKGDKYYLVPIYSWQVAKGILPDRAVVQGKDEEDWQLL | |
| | | | DDSFNFKFSLHPNDLVEVITKKARMFGYFASCHRGTGNINIRIHDLDHKIGKNGILEGIGVKT | |
| | | | ALSFQKYQIDELGKEIRPCRLKKRPPVR | |
| 18 | ABEMaxPI1 | | MKRTADGSEFESPKKKRKVGSDKKYSIGLAIGTNSVGWAVITDEYKVPSKKFKVLGNTDRHSI | |
| | | | KKNLIGALLFDSGETAEATRLKRTARRRYTRRKNRICYLQEIFSNEMAKVDDSFFHRLEESFLV | |
| | | | EEDKKHERHPIFGNIVDEVAYHEKYPTIYHLRKKLVDSTDKADLRLIYLALAHMIKFRGHFLIEG | |
| | | | DLNPDNSDVDKLFIQLVQTYNQLFEENPINASGVDAKAILSARLSKSRRLENLIAQLPGEKKN | |
| | | | GLFGNLIALSLGLTPNFKSNFDLAEDAKLQLSKDTYDDDLDNLLAQIGDQYADLFLAAKNLSD | |
| | | | AILLSDILRVNTEITKAPLSASMIKRYDEHHQDLTLLKALVRQQLPEKYKEIFFDQSKNGYAGYI | |
| | | | DGGASQEEFYKFIKPILEKMDGTEELLVKLNREDLLRKQRTFDNGSIPHQIHLGELHAILRRQE | |
| | | | DFYPFLKDNREKIEKILTFRIPYYVGPLARGNSRFAWMTRKSEETITPWNFEEVVDKGASAQS | |
| | | | FIERMTNFDKNLPNEKVLPKHSLLYEYFTVYNELTKVKYVTEGMRKPAFLSGEQKKAIVDLLF | |
| | | | KTNRKVTVKQLKEDYFKKIECFDSVEISGVEDRFNASLGTYHDLLKIIKDKDFLDNEENEDILED | |
| | | | IVLTLTLFEDREMIEERLKTYAHLFDDKVMKQLKRRRYTGWGRLSRKLINGIRDKQSGKTILDF | |
| | | | LKSDGFANRNFMQLIHDDSLTFKEDIQKAQVSGQGDSLHEHIANLAGSPAIKKGILQTVKVV | |
| | | | DELVKVMGRHKPENIVIEMARENQTTQKGQKNSRERMKRIEEGIKELGSQILKEHPVENTQ | |
| | | | LQNEKLYLYYLQNGRDMYVDQELDINRLSDYDVDHIVPQSFLKDDSIDNKVLTRSDKNRGKS | |
| | | | DNVPSEEVVKKMKNYWRQLLNAKLITQRKFDNLTKAERGGLSELDKAGFIKRQLVETRQITK | |
| | | | HVAQILDSRMNTKYDENDKLIREVKVITLKSKLVSDFRKDFQFYKVREINNYHHAHDAYLNA | |
| | | | VVGTALIKKYPKLESEFVYGDYKVYDVRKMIAKSEQEIGKATAKYFFYSNIMNFFKTEITLANG | |
| | | | EIRKRPLIETNGETGEIVWDKGRDFATVRKVLSMPQVNIVKKTEVQTGGFSKESILPKRNSDK | |
| | | | LIARKKDWDPKKYGGFDSPTVAYSVLVVAKVEKGKSKKLKSVKELLGITIMERSSFEKNPIDFL | |
| | | | EAKGYKEVKKDLIIKLPKYSLFELENGRKRMLASAGELQKGNELALPSKYVNFLYLASHYEKLK | |
| | | | GGSGGSGGSGGSGGSSEVEFSHEYWMRHALTLAKRARDEREVPVGAVLVLNNRVIGEGW | |
| | | | NRAIGLHDPTAHAEIMALRQGGLVMQNYRLIDATLYVTFEPCVMCAGAMIHSRIGRVVFG | |
| | | | VRNAKTGAAGSLMDVLHYPGMNHRVEITEGILADECAALLCYFFRMPRQVFNAQKKAQSS | |
| | | | TDGGSGGSGGSGGSGGSPEDNEQKQLFVEQHKHYLDEIIEQISEFSKRVILADANLDKVLSAY | |
| | | | NKHRDKPIREQAENIIHLFTLTNLGAPAAFKYFDTTIDRKRYTSTKEVLDATLIHQSITGLYETRI | |
| | | | DLSQLGGDSGGSKRTADGSEFESPKKKRKV | |
| 19 | ABEmaxPI2 | | MKRTADGSEFESPKKKRKVGSDKKYSIGLAIGTNSVGWAVITDEYKVPSKKFKVLGNTDRHSI | |
| | | | KKNLIGALLFDSGETAEATRLKRTARRRYTRRKNRICYLQEIFSNEMAKVDDSFFHRLEESFLV | |
| | | | EEDKKHERHPIFGNIVDEVAYHEKYPTIYHLRKKLVDSTDKADLRLIYLALAHMIKFRGHFLIEG | |
| | | | DLNPDNSDVDKLFIQLVQTYNQLFEENPINASGVDAKAILSARLSKSRRLENLIAQLPGEKKN | |
| | | | GLFGNLIALSLGLTPNFKSNFDLAEDAKLQLSKDTYDDDLDNLLAQIGDQYADLFLAAKNLSD | |
| | | | AILLSDILRVNTEITKAPLSASMIKRYDEHHQDLTLLKALVRQQLPEKYKEIFFDQSKNGYAGYI | |
| | | | DGGASQEEFYKFIKPILEKMDGTEELLVKLNREDLLRKQRTFDNGSIPHQIHLGELHAILRRQE | |
| | | | DFYPFLKDNREKIEKILTFRIPYYVGPLARGNSRFAWMTRKSEETITPWNFEEVVDKGASAQS | |
| | | | FIERMTNFDKNLPNEKVLPKHSLLYEYFTVYNELTKVKYVTEGMRKPAFLSGEQKKAIVDLLF | |
| | | | KTNRKVTVKQLKEDYFKKIECFDSVEISGVEDRFNASLGTYHDLLKIIKDKDFLDNEENEDILED | |
| | | | IVLTLTLFEDREMIEERLKTYAHLFDDKVMKQLKRRRYTGWGRLSRKLINGIRDKQSGKTILDF | |
| | | | LKSDGFANRNFMQLIHDDSLTFKEDIQKAQVSGQGDSLHEHIANLAGSPAIKKGILQTVKVV | |
| | | | DELVKVMGRHKPENIVIEMARENQTTQKGQKNSRERMKRIEEGIKELGSQILKEHPVENTQ | |
| | | | LQNEKLYLYYLQNGRDMYVDQELDINRLSDYDVDHIVPQSFLKDDSIDNKVLTRSDKNRGKS | |
| | | | DNVPSEEVVKKMKNYWRQLLNAKLITQRKFDNLTKAERGGLSELDKAGFIKRQLVETRQITK | |
| | | | HVAQILDSRMNTKYDENDKLIREVKVITLKSKLVSDFRKDFQFYKVREINNYHHAHDAYLNA | |
| | | | VVGTALIKKYPKLESEFVYGDYKVYDVRKMIAKSEQEIGKATAKYFFYSNIMNFFKTEITLANG | |
| | | | EIRKRPLIETNGETGEIVWDKGRDFATVRKVLSMPQVNIVKKTEVQTGGFSKESILPKRNSDK | |
| | | | LIARKKDWDPKKYGGFDSPTVAYSVLVVAKVEKGKSKKLKSVKELLGITIMERSSFEKNPIDFL | |
| | | | EAKGYKEVKKDLIIKLPKYSLFELENGRKRMLASAGELQKGNELALPSKYVNFLYLASHYEKLK | |
| | | | GGSGGSGGSSEVEFSHEYWMRHALTLAKRARDEREVPVGAVLVLNNRVIGEGWNRAIGLH | |
| | | | DPTAHAEIMALRQGGLVMQNYRLIDATLYVTFEPCVMCAGAMIHSRIGRVVFGVRNAKTG | |
| | | | AAGSLMDVLHYPGMNHRVEITEGILADECAALLCYFFRMPRQVFNAQKKAQSSTDGGSGG | |
| | | | SGGSGGSPEDNEQKQLFVEQHKHYLDEIIEQISEFSKRVILADANLDKVLSAYNKHRDKPIRE | |
| | | | QAENIIHLFTLTNLGAPAAFKYFDTTIDRKRYTSTKEVLDATLIHQSITGLYETRIDLSQLGGDS | |
| | | | GGSKRTADGSEFESPKKKRKV | |
| 20 | ABEmaxPI2 | | MKRTADGSEFESPKKKRKVGSDKKYSIGLAIGTNSVGWAVITDEYKVPSKKFKVLGNTDRHSI | |
| | | | KKNLIGALLFDSGETAEATRLKRTARRRYTRRKNRICYLQEIFSNEMAKVDDSFFHRLEESFLV | |
| | | | EEDKKHERHPIFGNIVDEVAYHEKYPTIYHLRKKLVDSTDKADLRLIYLALAHMIKFRGHFLIEG | |
| | | | DLNPDNSDVDKLFIQLVQTYNQLFEENPINASGVDAKAILSARLSKSRRLENLIAQLPGEKKN | |
| | | | GLFGNLIALSLGLTPNFKSNFDLAEDAKLQLSKDTYDDDLDNLLAQIGDQYADLFLAAKNLSD | |
| | | | AILLSDILRVNTEITKAPLSASMIKRYDEHHQDLTLLKALVRQQLPEKYKEIFFDQSKNGYAGYI | |
| | | | DGGASQEEFYKFIKPILEKMDGTEELLVKLNREDLLRKQRTFDNGSIPHQIHLGELHAILRRQE | |
| | | | DFYPFLKDNREKIEKILTFRIPYYVGPLARGNSRFAWMTRKSEETITPWNFEEVVDKGASAQS | |
| | | | FIERMTNFDKNLPNEKVLPKHSLLYEYFTVYNELTKVKYVTEGMRKPAFLSGEQKKAIVDLLF | |
| | | | KTNRKVTVKQLKEDYFKKIECFDSVEISGVEDRFNASLGTYHDLLKIIKDKDFLDNEENEDILED | |
| | | | IVLTLTLFEDREMIEERLKTYAHLFDDKVMKQLKRRRYTGWGRLSRKLINGIRDKQSGKTILDF | |
| | | | LKSDGFANRNFMQLIHDDSLTFKEDIQKAQVSGQGDSLHEHIANLAGSPAIKKGILQTVKVV | |
| | | | DELVKVMGRHKPENIVIEMARENQTTQKGQKNSRERMKRIEEGIKELGSQILKEHPVENTQ | |
| | | | LQNEKLYLYYLQNGRDMYVDQELDINRLSDYDVDHIVPQSFLKDDSIDNKVLTRSDKNRGKS | |
| | | | DNVPSEEVVKKMKNYWRQLLNAKLITQRKFDNLTKAERGGLSELDKAGFIKRQLVETRQITK | |
| | | | HVAQILDSRMNTKYDENDKLIREVKVITLKSKLVSDFRKDFQFYKVREINNYHHAHDAYLNA | |
| | | | VVGTALIKKYPKLESEFVYGDYKVYDVRKMIAKSEQEIGKATAKYFFYSNIMNFFKTEITLANG | |
| | | | EIRKRPLIETNGETGEIVWDKGRDFATVRKVLSMPQVNIVKKTEVQTGGFSKESILPKRNSDK | |
| | | | LIARKKDWDPKKYGGFDSPTVAYSVLVVAKVEKGKSKKLKSVKELLGITIMERSSFEKNPIDFL | |
| | | | EAKGYKEVKKDLIIKLPKYSLFELENGRKRMLASAGELQKGNELALPSKYVNFLYLASHYEKLK | |
| | | | GGSGGSGGSSEVEFSHEYWMRHALTLAKRARDEREVPVGAVLVLNNRVIGEGWNRAIGLH | |
| | | | DPTAHAEIMALRQGGLVMQNYRLIDATLYVTFEPCVMCAGAMIHSRIGRVVFGVRNAKTG | |
| | | | AAGSLMDVLHYPGMNHRVEITEGILADECAALLCYFFRMPRQVFNAQKKAQSSTDGGSGG | |
| | | | SGGSPEDNEQKQLFVEQHKHYLDEIIEQISEFSKRVILADANLDKVLSAYNKHRDKPIREQAE | |
| | | | NIIHLFTLTNLGAPAAFKYFDTTIDRKRYTSTKEVLDATLIHQSITGLYETRIDLSQLGGDSGGSK | |
| | | | RTADGSEFESPKKKRKV | |
| 21 | ABEmax | | MKRTADGSEFESPKKKRKVSEVEFSHEYWMRHALTLAKRAWDEREVPVGAVLVHNNRVIG | |
| | | | EGWNRPIGRHDPTAHAEIMALRQGGLVMQNYRLIDATLYVTLEPCVMCAGAMIHSRIGRV | |
| | | | VFGARDAKTGAAGSLMDVLHHPGMNHRVEITEGILADECAALLSDFFRMRRQEIKAQKKA | |
| | | | QSSTDSGGSSGGSSGSETPGTSESATPESSGGSSGGSSEVEFSHEYWMRHALTLAKRARDER | |
| | | | EVPVGAVLVLNNRVIGEGWNRAIGLHDPTAHAEIMALRQGGLVMQNYRLIDATLYVTFEP | |
| | | | CVMCAGAMIHSRIGRVVFGVRNAKTGAAGSLMDVLHYPGMNHRVEITEGILADECAALLC | |
| | | | YFFRMPRQVFNAQKKAQSSTDSGGSSGGSSGSETPGTSESATPESSGGSSGGSDKKYSIGLAI | |
| | | | GTNSVGWAVITDEYKVPSKKFKVLGNTDRHSIKKNLIGALLFDSGETAEATRLKRTARRRYTR | |
| | | | RKNRICYLQEIFSNEMAKVDDSFFHRLEESFLVEEDKKHERHPIFGNIVDEVAYHEKYPTIYHL | |
| | | | RKKLVDSTDKADLRLIYLALAHMIKFRGHFLIEGDLNPDNSDVDKLFIQLVQTYNQLFEENPIN | |
| | | | ASGVDAKAILSARLSKSRRLENLIAQLPGEKKNGLFGNLIALSLGLTPNFKSNFDLAEDAKLQL | |
| | | | SKDTYDDDLDNLLAQIGDQYADLFLAAKNLSDAILLSDILRVNTEITKAPLSASMIKRYDEHHQ | |
| | | | DLTLLKALVRQQLPEKYKEIFFDQSKNGYAGYIDGGASQEEFYKFIKPILEKMDGTEELLVKLN | |
| | | | REDLLRKQRTFDNGSIPHQIHLGELHAILRRQEDFYPFLKDNREKIEKILTFRIPYYVGPLARGN | |
| | | | SRFAWMTRKSEETITPWNFEEVVDKGASAQSFIERMTNFDKNLPNEKVLPKHSLLYEYFTVY | |
| | | | NELTKVKYVTEGMRKPAFLSGEQKKAIVDLLFKTNRKVTVKQLKEDYFKKIECFDSVEISGVED | |
| | | | RFNASLGTYHDLLKIIKDKDFLDNEENEDILEDIVLTLTLFEDREMIEERLKTYAHLFDDKVMK | |
| | | | QLKRRRYTGWGRLSRKLINGIRDKQSGKTILDFLKSDGFANRNFMQLIHDDSLTFKEDIQOKA | |
| | | | QVSGQGDSLHEHIANLAGSPAIKKGILQTVKVVDELVKVMGRHKPENIVIEMARENQTTQK | |
| | | | GQKNSRERMKRIEEGIKELGSQILKEHPVENTQLQNEKLYLYYLQNGRDMYVDQELDINRLS | |
| | | | DYDVDHIVPQSFLKDDSIDNKVLTRSDKNRGKSDNVPSEEVVKKMKNYWRQLLNAKLITQR | |
| | | | KFDNLTKAERGGLSELDKAGFIKRQLVETRQITKHVAQILDSRMNTKYDENDKLIREVKVITLK | |
| | | | SKLVSDFRKDFQFYKVREINNYHHAHDAYLNAVVGTALIKKYPKLESEFVYGDYKVYDVRKM | |
| | | | IAKSEQEIGKATAKYFFYSNIMNFFKTEITLANGEIRKRPLIETNGETGEIVWDKGRDFATVRK | |
| | | | VLSMPQVNIVKKTEVQTGGFSKESILPKRNSDKLIARKKDWDPKKYGGFDSPTVAYSVLVVA | |
| | | | KVEKGKSKKLKSVKELLGITIMERSSFEKNPIDFLEAKGYKEVKKDLIIKLPKYSLFELENGRKRM | |
| | | | LASAGELQKGNELALPSKYVNFLYLASHYEKLKGSPEDNEQKQLFVEQHKHYLDEIIEQISEFS | |
| | | | KRVILADANLDKVLSAYNKHRDKPIREQAENIIHLFTLTNLGAPAAFKYFDTTIDRKRYTSTKE | |
| | | | VLDATLIHQSITGLYETRIDLSQLGGDSGGSKRTADGSEFESPKKKRKV | |
| 22 | dABEmax | | MKRTADGSEFESPKKKRKVSEVEFSHEYWMRHALTLAKRAWDEREVPVGAVLVHNNRVIG | H840A |
| | | | EGWNRPIGRHDPTAHAEIMALRQGGLVMQNYRLIDATLYVTLEPCVMCAGAMIHSRIGRV | |
| | | | VFGARDAKTGAAGSLMDVLHHPGMNHRVEITEGILADECAALLSDFFRMRROEIKAOKKA | |
| | | | QSSTDSGGSSGGSSGSETPGTSESATPESSGGSSGGSSEVEFSHEYWMRHALTLAKRARDER | |
| | | | EVPVGAVLVLNNRVIGEGWNRAIGLHDPTAHAEIMALRQGGLVMQNYRLIDATLYVTFEP | |
| | | | CVMCAGAMIHSRIGRVVFGVRNAKTGAAGSLMDVLHYPGMNHRVEITEGILADECAALLC | |
| | | | YFFRMPRQVFNAQKKAQSSTDSGGSSGGSSGSETPGTSESATPESSGGSSGGSDKKYSIGLAI | |
| | | | GTNSVGWAVITDEYKVPSKKFKVLGNTDRHSIKKNLIGALLFDSGETAEATRLKRTARRRYTR | |
| | | | RKNRICYLQEIFSNEMAKVDDSFFHRLEESFLVEEDKKHERHPIFGNIVDEVAYHEKYPTIYHL | |
| | | | RKKLVDSTDKADLRLIYLALAHMIKFRGHFLIEGDLNPDNSDVDKLFIQLVQTYNQLFEENPIN | |
| | | | ASGVDAKAILSARLSKSRRLENLIAQLPGEKKNGLFGNLIALSLGLTPNFKSNFDLAEDAKLQL | |
| | | | SKDTYDDDLDNLLAQIGDQYADLFLAAKNLSDAILLSDILRVNTEITKAPLSASMIKRYDEHHQ | |
| | | | DLTLLKALVRQQLPEKYKEIFFDQSKNGYAGYIDGGASQEEFYKFIKPILEKMDGTEELLVKLN | |
| | | | REDLLRKQRTFDNGSIPHQIHLGELHAILRRQEDFYPFLKDNREKIEKILTFRIPYYVGPLARGN | |
| | | | SRFAWMTRKSEETITPWNFEEVVDKGASAQSFIERMTNFDKNLPNEKVLPKHSLLYEYFTVY | |
| | | | NELTKVKYVTEGMRKPAFLSGEQKKAIVDLLFKTNRKVTVKQLKEDYFKKIECFDSVEISGVED | |
| | | | RFNASLGTYHDLLKIIKDKDFLDNEENEDILEDIVLTLTLFEDREMIEERLKTYAHLFDDKVMK | |
| | | | QLKRRRYTGWGRLSRKLINGIRDKQSGKTILDFLKSDGFANRNFMQLIHDDSLTFKEDIQOKA | |
| | | | QVSGQGDSLHEHIANLAGSPAIKKGILQTVKVVDELVKVMGRHKPENIVIEMARENQTTQK | |
| | | | GQKNSRERMKRIEEGIKELGSQILKEHPVENTQLQNEKLYLYYLQNGRDMYVDQELDINRLS | |
| | | | DYDVDAIVPQSFLKDDSIDNKVLTRSDKNRGKSDNVPSEEVVKKMKNYWRQLLNAKLITQR | |
| | | | KFDNLTKAERGGLSELDKAGFIKRQLVETRQITKHVAQILDSRMNTKYDENDKLIREVKVITLK | |
| | | | SKLVSDFRKDFQFYKVREINNYHHAHDAYLNAVVGTALIKKYPKLESEFVYGDYKVYDVRKM | |
| | | | IAKSEQEIGKATAKYFFYSNIMNFFKTEITLANGEIRKRPLIETNGETGEIVWDKGRDFATVRK | |
| | | | VLSMPQVNIVKKTEVQTGGFSKESILPKRNSDKLIARKKDWDPKKYGGFDSPTVAYSVLVVA | |
| | | | KVEKGKSKKLKSVKELLGITIMERSSFEKNPIDFLEAKGYKEVKKDLIIKLPKYSLFELENGRKRM | |
| | | | LASAGELQKGNELALPSKYVNFLYLASHYEKLKGSPEDNEQKQLFVEQHKHYLDEIIEQISEFS | |
| | | | KRVILADANLDKVLSAYNKHRDKPIREQAENIIHLFTLTNLGAPAAFKYFDTTIDRKRYTSTKE | |
| | | | VLDATLIHQSITGLYETRIDLSQLGGDSGGSKRTADGSEFESPKKKRKV | |
| 23 | ABEmaxΔHNH | | MKRTADGSEFESPKKKRKVSEVEFSHEYWMRHALTLAKRAWDEREVPVGAVLVHNNRVIG | |
| | | | EGWNRPIGRHDPTAHAEIMALRQGGLVMQNYRLIDATLYVTLEPCVMCAGAMIHSRIGRV | |
| | | | VFGARDAKTGAAGSLMDVLHHPGMNHRVEITEGILADECAALLSDFFRMRRQEIKAQKKA | |
| | | | QSSTDSGGSSGGSSGSETPGTSESATPESSGGSSGGSSEVEFSHEYWMRHALTLAKRARDER | |
| | | | EVPVGAVLVLNNRVIGEGWNRAIGLHDPTAHAEIMALRQGGLVMQNYRLIDATLYVTFEP | |
| | | | CVMCAGAMIHSRIGRWFGVRNAKTGAAGSLMDVLHYPGMNHRVEITEGILADECAALLC | |
| | | | YFFRMPRQVFNAQKKAQSSTDSGGSSGGSSGSETPGTSESATPESSGGSSGGSDKKYSIGLAI | |
| | | | GTNSVGWAVITDEYKVPSKKFKVLGNTDRHSIKKNLIGALLFDSGETAEATRLKRTARRRYTR | |
| | | | RKNRICYLQEIFSNEMAKVDDSFFHRLEESFLVEEDKKHERHPIFGNIVDEVAYHEKYPTIYHL | |
| | | | RKKLVDSTDKADLRLIYLALAHMIKFRGHFLIEGDLNPDNSDVDKLFIQLVQTYNQLFEENPIN | |
| | | | ASGVDAKAILSARLSKSRRLENLIAQLPGEKKNGLFGNLIALSLGLTPNFKSNFDLAEDAKLQL | |
| | | | SKDTYDDDLDNLLAQIGDQYADLFLAAKNLSDAILLSDILRVNTEITKAPLSASMIKRYDEHHQ | |
| | | | DLTLLKALVRQQLPEKYKEIFFDQSKNGYAGYIDGGASQEEFYKFIKPILEKMDGTEELLVKLN | |
| | | | REDLLRKQRTFDNGSIPHQIHLGELHAILRRQEDFYPFLKDNREKIEKILTFRIPYYVGPLARGN | |
| | | | SRFAWMTRKSEETITPWNFEEVVDKGASAQSFIERMTNFDKNLPNEKVLPKHSLLYEYFTVY | |
| | | | NELTKVKYVTEGMRKPAFLSGEQKKAIVDLLFKTNRKVTVKQLKEDYFKKIECFDSVEISGVED | |
| | | | RFNASLGTYHDLLKIIKDKDFLDNEENEDILEDIVLTLTLFEDREMIEERLKTYAHLFDDKVMK | |
| | | | QLKRRRYTGWGRLSRKLINGIRDKQSGKTILDFLKSDGFANRNFMQLIHDDSLTFKEDIQOKA | |
| | | | QVSGQGDSLHEHIANLAGSPAIKKGILQTVKWDELVKVMGRHKPENMEMARENQGGSG | |
| | | | GSRQLVETRQITKHVAQILDSRMNTKYDENDKLIREVKVITLKSKLVSDFRKDFQFYKVREIN | |
| | | | NYHHAHDAYLNAVVGTALIKKYPKLESEFVYGDYKVYDVRKMIAKSEQEIGKATAKYFFYSNI | |
| | | | MNFFKTEITLANGEIRKRPLIETNGETGEIVWDKGRDFATVRKVLSMPQVNIVKKTEVQTGG | |
| | | | FSKESILPKRNSDKLIARKKDWDPKKYGGFDSPTVAYSVLVVAKVEKGKSKKLKSVKELLGITI | |
| | | | MERSSFEKNPIDFLEAKGYKEVKKDLIIKLPKYSLFELENGRKRMLASAGELQKGNELALPSKY | |
| | | | VNFLYLASHYEKLKGSPEDNEQKQLFVEQHKHYLDEIIEQISEFSKRVILADANLDKVLSAYNK | |
| | | | HRDKPIREQAENIIHLFTLTNLGAPAAFKYFDTTIDRKRYTSTKEVLDATLIHQSITGLYETRIDL | |
| | | | SQLGGDSGGSKRTADGSEFESPKKKRKV | |
| 24 | NLS | | MKRTADGSEFEPKKKRKV | |
| 25 | NLS | | KRTADGSEFEPKKKRKV | |
| 26 | | | MSEVEFSHEYWMRHALTLAKRARDEREVPVGAVLVHNNRVIGEGWNRPIGRHDPTAHAE | A106V, D108N, |
| | | | IMALRQGGLVMQNYRLIDATLYVTLEPCVMCAGAMIHSRIGRVVFGARDAKTGAAGSLM | D147Y, L84F, |
| | | | DVLHHPGMNHRVEITEGILADECAALLSDFFRMRRQEIKAQKKAQSSTD | H123Y, I157F, |
| | | | | and/or E155V |

What is claimed is:

1. A chimeric enzyme comprising a CRISPR class 2 type II enzyme backbone, wherein the HNH domain in the backbone has been replaced, essentially, by a peptide or protein domain having catalytic activity on a single stranded polynucleotide.

2. The chimeric enzyme according to claim 1, wherein the peptide or protein domain having catalytic activity on a single stranded nucleotide is a peptide or protein domain having at least one selected from the group consisting of a) deaminase activity,
b) reverse transcriptase activity,
c) methyltransferase activity,
d) transposase activity,
e) polymerase activity, and
f) nuclease activity

3. The chimeric enzyme according to claim 1 or 2, wherein the CRISPR class 2 type II enzyme backbone is a CRISPR Cas9 enzyme backbone.

4. The chimeric enzyme according to claim 3, wherein the CRISPR Cas9 enzyme backbone is a backbone taken from one member of the group consisting of
SaCas9,
SpCas9,
StCas9,
CjCas9, and
NmeCas9.

5. The chimeric enzyme according to claim 4, wherein the CRISPR Cas9 enzyme backbone comprises
a) an amino acid sequence set forth in SEQ ID NO 1, SEQ ID NO 2, SEQ ID NO 3, SEQ ID NO 16 or SEQ ID NO 17, or
b) an amino acid sequence having at least 80% sequence identity therewith.

6. The chimeric enzyme according to any one of the aforementioned claims, wherein the CRISPR Cas9 enzyme backbone is catalytically inactive and/or lacks endonuclease activity.

7. The chimeric enzyme according to any one of the aforementioned claims, wherein the deaminase catalyzes
a) deamination of cytosine, or
b) deamination of adenosine.

8. The chimeric enzyme according to any one of the aforementioned claims, wherein the deaminase comprises at least one of the enzymes selected from the group consisting of
apolipoprotein B mRNA-editing complex (APOBEC) deaminase
cytidine deaminase, and/or
adenosine deaminase
or at least a catalytically active domain derived therefrom maintaining deaminase activity.

9. The chimeric enzyme according to claiml, wherein the deaminase comprises a sequence selected from
a) the group consisting of enzymes selected from the group consisting of SEQ ID NO 4-7, or
b) a sequence having at least 80% sequence identity with SEQ ID NO 4-7 while maintaining deaminase activity, or
c) a catalytically active domain derived from the deaminase of a) or b), with the optional proviso that
SEQ ID NO 4 has at least one amino acid substitution selected from the group consisting of D108N, A106V, D147Y, E155V, L84F, H123Y, and/or I157F, and/or
SEQ ID NO 6 has at least one amino acid substitution selected from the group consisting of F22S, A123V, and/or I195F.

10. The chimeric enzyme according to any one of the aforementioned claims, wherein the reverse transcriptase comprises M-MLV RT (Moloney Murine Leukemia Virus Reverse Transcriptase) or at least a catalytically active domain derived therefrom maintaining reverse transcriptase activity.

11. The chimeric enzyme according to any one of the aforementioned claims, wherein the reverse transcriptase comprises an amino acid sequence selected from
a) SEQ ID NO 15, or
b) a sequence having at least 80% sequence identity with SEQ ID NO 15 while reverse transcriptase activity, or
c) a catalytically active domain derived from reverse transcriptase of a) or b), with the optional proviso that SEQ ID NO 15 has at least one amino acid substitution selected from the group consisting of D200N, T306K, W313F, T330P, L603W

12. The chimeric enzyme according to any one of the aforementioned claims, which enzyme further comprises
a) at least one nuclear localization sequence (NLS), and/or
b) at least one inhibitor of nucleic acid repair, preferably a Uracil-DNA glycosylase inhibitor (UGI)

13. The chimeric enzyme according to any one of the aforementioned claims, which enzyme has the following domain structure, shown in N->C direction:
RuvC-I— Recognition lobe—RuvC-II— deaminase—RuvC-III— PI or
RuvC-I— Recognition lobe—RuvC-II— reverse transcriptase—RuvC-III— PI or
RuvC-I— Recognition lobe—RuvC-II— methyltransferase—RuvC-III— PI or
RuvC-I— Recognition lobe—RuvC-II— transposase—RuvC-III— PI or
RuvC-I— Recognition lobe—RuvC-II— polymerase—RuvC-III— PI or
RuvC-I— Recognition lobe—RuvC-II— nuclease—RuvC-III— PI
with “—” being optional linkers, and optionally
(iii) a nuclear localization sequence (NLS) at the C-terminus and/or the N-terminus and/or
(iv) at least one Uracil-DNA glycosylase inhibitor (UGI) domain at the N-terminus.

14. The chimeric enzyme according to any one of the aforementioned claims, which enzyme comprises an amino acid sequence according to SEQ ID NOs 12-14

15. A nucleic acid encoding for the enzyme of any one of claims 1-14

16. A vector comprising the nucleic acid according to claim 15

17. A combination comprising the enzyme of any one of claims 1-14, or the nucleic acid of claim 15, or the vector of claim 16, and at least one of
a) a combination of a crRNA and a tracrRNA,
b) a single guide RNA, and/or
c) a pegRNA

18. A method for editing a nucleobase and/or reversing a single nucleotide polymorphism within a nucleotide sequence, the method comprising:
a) contacting said nucleotide sequence with the combination of claim 17, and
b) converting a first nucleobase of said nucleotide sequence to a second nucleobase, or reversing the single nucleotide polymorphism.

19. The method according to claim 18, wherein said first nucleobase is adenine or guanine, and said second nucleobase is inosine or uracil.

20. The method according to claim 18-19, wherein a third nucleobase complementary to said first nucleobase is replaced by a fourth nucleobase complementary to said second nucleobase.

21. The method according to any one of claims 18-20, wherein the contacting takes place ex vivo/in vitro, or in vivo.


SEQUENCE LISTING (26 sequences)

SEQ ID NO: 1 - protein, 1368 aa, Streptococcus pyogenes [sequence omitted]

SEQ ID NO: 2 - protein, 1053 aa, Staphylococcus aureus
MKRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 3 - protein, 1409 aa, Streptococcus thermophilus
MLFNKCIIISINLDFSNKEKCMTKPYSIGLDIGTNSVGWAVITDNYKVPSKKMKVLGNTSKKYIKKNLLGVLLFDSGITAEGRRLKRTARRRYTRRRNRILYLQEIFSTEMATLDDAFFQRLDDSFLVPDDKRDSKYPIFGNLVEEKVYHDEFPTIYHLRKYLADSTKKADLRLVYLALAHMIKYRGHFLIEGEFNSKNNDIQKNFQDFLDTYNAIFESDLSLENSKQLEEIVKDKISKLEKKDRILKLFPGEKNSGIFSEFLKLIVGNQADFRKCFNLDEKASLHFSKESYDEDLETLLGYIGDDYSDVFLKAKKLYDAILLSGFLTVTDNETEAPLSSAMIKRYNEHKEDLALLKEYIRNISLKTYNEVFKDDTKNGYAGYIDGKTNQEDFYVYLKNLLAEFEGADYFLEKIDREDFLRKQRTFDNGSIPYQIHLQEMRAILDKQAKFYPFLAKNKERIEKILTFRIPYYVGPLARGNSDFAWSIRKRNEKITPWNFEDVIDKESSAEAFINRMTSFDLYLPEEKVLPKHSLLYETFNVYNELTKVRFIAESMRDYQFLDSKQKKDIVRLYFKDKRKVTDKDIIEYLHAIYGYDGIELKGIEKQFNSSLSTYHDLLNIINDKEFLDDSSNEAIIEEIIHTLTIFEDREMIKQRLSKFENIFDKSVLKKLSRRHYTGWGKLSAKLINGIRDEKSGNTILDYLIDDGISNRNFMQLIHDDALSFKKKIQKAQIIGDEDKGNIKEVVKSLPGSPAIKKGILQSIKIVDELVKVMGGRKPESIVVEMARENQYTNQGKSNSQQRLKRLEKSLKELGSKILKENIPAKLSKIDNNALQNDRLYLYYLQNGKDMYTGDDLDIDRLSNYDIDHIIPQAFLKDNSIDNKVLVSSASNRGKSDDFPSLEVVKKRKTFWYQLLKSKLISQRKFDNLTKAERGGLLPEDKAGFIQRQLVETRQITKHVARLLDEKFNNKKDENNRAVRTVKIITLKSTLVSQFRKDFELYKVREINDFHHAHDAYLNAVIASALLKKYPKLEPEFVYGDYPKYNSFRERKSATEKVYFYSNIMNIFKKSISLADGRVIERPLIEVNEETGESVWNKESDLATVRRVLSYPQVNVVKKVEEQNHGLDRGKPKGLFNANLSSKPKPNSNENLVGAKEYLDPKKYGGYAGISNSFAVLVKGTIEKGAKKKITNVLEFQGISILDRINYRKDKLNFLLEKGYKDIELIIELPKYSLFELSDGSRRMLASILSTNNKRGEIHKGNQIFLSQKFVKLLYHAKRISNTINENHRKYVENHKKEFEELFYYILEFNENYVGAKKNGKLLNSAFQSWQNHSIDELCSSFIGPTGSERKGLFELTSRGSAADFEFLGVKIPRYRDYTPSSLLKDATLIHQSVTGLYETRIDLAKLGEG

SEQ ID NO: 4 - protein, 167 aa, E. coli
MSEVEFSHEYWMRHALTLAKRAWDEREVPVGAVLVHNNRVIGEGWNRPIGRHDPTAHAEIMALRQGGLVMQNYRLIDATLYVTLEPCVMCAGAMIHSRIGRVVFGARDAKTGAAGSLMDVLHHPGMNHRVEITEGILADECAALLSDFFRMRRQEIKAQKKAQSSTD

SEQ ID NO: 5 - protein, 236 aa, Homo sapiens
MTSEKGPSTGDPTLRRRIEPWEFDVFYDPRELRKEACLLYEIKWGMSRKIWRSSGKNTTNHVEVNFIKKFTSERDFHPSMSCSITWFLSWSPCWECSQAIREFLSRHPGVTLVIYVARLFWHMDQQNRQGLRDLVNSGVTIQIMRASEYYHCWRNFVNYPPGDEAHWPQYPPLWMMLYALELHCIILSLPPCLKISRRWQNHLTFFRLHLQNCHYQTIPPHILLATGLIHPSVAWR

SEQ ID NO: 6 - protein, 208 aa, Petromyzon marinus
MTDAEYVRIHEKLDIYTFKKQFFNNKKSVSHRCYVLFELKRRGERRACFWGYAVNKPQSGTERGIHAEIFSIRKVEEYLRDNPGQFTINWYSSWSPCADCAEKILEWYNQELRGNGHTLKIWACKLYYEKNARNQIGLWNLRDNGVGLNVMVSEHYQCCRKIFIQSSHNQLNENRWLEKTLKRAEKRRSELSIMIQVKILHTTKSPAV

SEQ ID NO: 7 - protein, 161 aa, artificial sequence
  Ferny deaminase
FERNYDPRELRKETYLLYEIKWGKSGKLWRHWCQNNRTQHAEVYFLENIFNARRFNPSTHCSITWYLSWSPCAECSQKIVDFLKEHPNVNLEIYVARLYYPENERNRQGLRDLVNSGVTIRIMDLPDYNYCWKTFVSDQGGDEDYWPGHFAPWIKQYSLKL

SEQ ID NO: 8 - protein, 19 aa, artificial sequence
  NLS
MKRTADGSEFESPKKKRKV

SEQ ID NO: 9 - protein, 18 aa, artificial sequence
  NLS
KRTADGSEFESPKKKRKV

SEQ ID NO: 10 - protein, 83 aa, Bacillus phage PBS2
TNLSDIIEKETGKQLVIQESILMLPEEVEEVIGNKPESDILVHTAYDESTDENVMLLTSDAPEYKPWALVIQDSNGENKIKML

SEQ ID NO: 11 - protein, 176 aa, artificial sequence
  UGI
TNLSDIIEKETGKQLVIQESILMLPEEVEEVIGNKPESDILVHTAYDESTDENVMLLTSDAPEYKPWALVIQDSNGENKIKMLSGGSGGSGGSTNLSDIIEKETGKQLVIQESILMLPEEVEEVIGNKPESDILVHTAYDESTDENVMLLTSDAPEYKPWALVIQDSNGENKIKML

SEQ ID NO: 12 - protein, 1457 aa, artificial sequence
  HNHx_TadA_ABE
MKRTADGSEFESPKKKRKVGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSGGSSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNAKTGAAGSLMDVLHYPGMNHRVEITEGILADECAALLCYFFRMPRQVFNAQKKAQSSTDSGGRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSKRTADGSEFESPKKKRKV

SEQ ID NO: 13 - protein, 1626 aa, artificial sequence
  HNHx_FERNY_ABE
MKRTADGSEFESPKKKRKVGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGSGGSGSFERNYDPRELRKETYLLYEIKWGKSGKLWRHWCQNNRTQHAEVYFLENIFNARRFNPSTHCSITWYLSWSPCAECSQKIVDFLKEHPNVNLEIYVARLYYPENERNRQGLRDLVNSGVTIRIMDLPDYNYCWKTFVSDQGGDEDYWPGHFAPWIKQYSLKLSGKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSGGSGGSTNLSDIIEKETGKQLVIQESILMLPEEVEEVIGNKPESDILVHTAYDESTDENVMLLTSDAPEYKPWALVIQDSNGENKIKMLSGGSGGSGGSTNLSDIIEKETGKQLVIQESILMLPEEVEEVIGNKPESDILVHTAYDESTDENVMLLTSDAPEYKPWALVIQDSNGENKIKMLSGGSKRTADGSEFESPKKKRKV

SEQ ID NO: 14 - protein, 1684 aa, artificial sequence
  HNHx_pmCDA1_ABE
MKRTADGSEFESPKKKRKVGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSTDAEYVRIHEKLDIYTFKKQFSNNKKSVSHRCYVLFELKRRGERRACFWGYAVNKPQSGTERGIHAEIFSIRKVEEYLRDNPGQFTINWYSSWSPCADCAEKILEWYNQELRGNGHTLKIWVCKLYYEKNARNQIGLWNLRDNGVGLNVMVSEHYQCCRKIFIQSSHNQLNENRWLEKTLKRAEKRRSELSIMFQVKILHTTKSPAVSGGSGGRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSGGSGGSTNLSDIIEKETGKQLVIQESILMLPEEVEEVIGNKPESDILVHTAYDESTDENVMLLTSDAPEYKPWALVIQDSNGENKIKMLSGGSGGSGGSTNLSDIIEKETGKQLVIQESILMLPEEVEEVIGNKPESDILVHTAYDESTDENVMLLTSDAPEYKPWALVIQDSNGENKIKMLSGGSKRTADGSEFESPKKKRKV

SEQ ID NO: 15 - protein, 677 aa, Moloney murine leukaemia virus
TLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFDEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGTAGFCRLWIPGFAEMAAPLYPLTKTGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGLLTSEGKEIKNKDEILALLKALFLPKRLSIIHCPGHQKGHSAEARGNRMADQAARKAAITETPDTSTLLIENSSP

SEQ ID NO: 16 - protein, 984 aa, Campylobacter jejuni
MARILAFDIGISSIGWAFSENDELKDCGVRIFTKVENPKTGESLALPRRLARSARKRLARRKARLNHLKHLIANEFKLNYEDYQSFDESLAKAYKGSLISPYELRFRALNELLSKQDFARVILHIAKRRGYDDIKNSDDKEKGAILKAIKQNEEKLANYQSVGEYLYKEYFQKFKENSKEFTNVRNKKESYERCIAQSFLKDELKLIFKKQREFGFSFSKKFEEEVLSVAFYKRALKDFSHLVGNCSFFTDEKRAPKNSPLAFMFVALTRIINLLNNLKNTEGILYTKDDLNALLNEVLKNGTLTYKQTKKLLGLSDDYEFKGEKGTYFIEFKKYKEFIKALGEHNLSQDDLNEIAKDITLIKDEIKLKKALAKYDLNQNQIDSLSKLEFKDHLNISFKALKLVTPLMLEGKKYDEACNELNLKVAINEDKKDFLPAFNETYYKDEVTNPVVLRAIKEYRKVLNALLKKYGKVHKINIELAREVGKNHSQRAKIEKEQNENYKAKKDAELECEKLGLKINSKNILKLRLFKEQKEFCAYSGEKIKISDLQDEKMLEIDHIYPYSRSFDDSYMNKVLVFTKQNQEKLNQTPFEAFGNDSAKWQKIEVLAKNLPTKKQKRILDKNYKDKEQKNFKDRNLNDTRYIARLVLNYTKDYLDFLPLSDDENTKLNDTQKGSKVHVEAKSGMLTSALRHTWGFSAKDRNNHLHHAIDAVIIAYANNSIVKAFSDFKKEQESNSAELYAKKISELDYKNKRKFFEPFSGFRQKVLDKIDEIFVSKPERKKPSGALHEETFRKEEEFYQSYGGKEGVLKALELGKIRKVNGKIVKNGDMFRVDIFKHKKTNKFYAVPIYTMDFALKVLPNKAVARSKKGEIKDWILMDENYEFCFSLYKDSLILIQTKDMQEPEFVYYNAFTSSTVSLIVSKHDNKFETLSKNQKILFKNANEKEVIAKSIGIQNLKVFEKYIVSALGEVTKAEFRQREDFKK

SEQ ID NO: 17 - protein, 1082 aa, Neisseria meningitidis
MAAFKPNPINYILGLDIGIASVGWAMVEIDEDENPICLIDLGVRVFERAEVPKTGDSLAMARRLARSVRRLTRRRAHRLLRARRLLKREGVLQAADFDENGLIKSLPNTPWQLRAAALDRKLTPLEWSAVLLHLIKHRGYLSQRKNEGETADKELGALLKGVADNAHALQTGDFRTPAELALNKFEKESGHIRNQRGDYSHTFSRKDLQAELILLFEKQKEFGNPHVSGGLKEGIETLLMTQRPALSGDAVQKMLGHCTFEPAEPKAAKNTYTAERFIWLTKLNNLRILEQGSERPLTDTERATLMDEPYRKSKLTYAQARKLLGLEDTAFFKGLRYGKDNAEASTLMEMKAYHAISRALEKEGLKDKKSPLNLSPELQDEIGTAFSLFKTDEDITGRLKDRIQPEILEALLKHISFDKFVQISLKALRRIVPLMEQGKRYDEACAEIYGDHYGKKNTEEKIYLPPIPADEIRNPVVLRALSQARKVINGVVRRYGSPARIHIETAREVGKSFKDRKEIEKRQEENRKDREKAAAKFREYFPNFVGEPKSKDILKLRLYEQQHGKCLYSGKEINLGRLNEKGYVEIDHALPFSRTWDDSFNNKVLVLGSENQNKGNQTPYEYFNGKDNSREWQEFKARVETSRFPRSKKQRILLQKFDEDGFKERNLNDTRYVNRFLCQFVADRMRLTGKGKKRVFASNGQITNLLRGFWGLRKVRAENDRHHALDAVVVACSTVAMQQKITRFVRYKEMNAFDGKTIDKETGEVLHQKTHFPQPWEFFAQEVMIRVFGKPDGKPEFEEADTPEKLRTLLAEKLSSRPEAVHEYVTPLFVSRAPNRKMSGQGHMETVKSAKRLDEGVSVLRVPLTQLKLKDLEKMVNREREPKLYEALKARLEAHKDDPAKAFAEPFYKYDKAGNRTQQVKAVRVEQVQKTGVWVRNHNGIADNATMVRVDVFEKGDKYYLVPIYSWQVAKGILPDRAVVQGKDEEDWQLIDDSFNFKFSLHPNDLVEVITKKARMFGYFASCHRGTGNINIRIHDLDHKIGKNGILEGIGVKTALSFQKYQIDELGKEIRPCRLKKRPPVR

SEQ ID NO: 18 - protein, 1604 aa, artificial sequence
  ABEMaxPI1
MKRTADGSEFESPKKKRKVGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGGSGGSGGSGGSGGSSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNAKTGAAGSLMDVLHYPGMNHRVEITEGILADECAALLCYFFRMPRQVFNAQKKAQSSTDGGSGGSGGSGGSGGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSKRTADGSEFESPKKKRKV

SEQ ID NO: 19 - protein, 1595 aa, artificial sequence
  ABEMaxPI2
MKRTADGSEFESPKKKRKVGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGGSGGSGGSSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNAKTGAAGSLMDVLHYPGMNHRVEITEGILADECAALLCYFFRMPRQVFNAQKKAQSSTDGGSGGSGGSGGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSKRTADGSEFESPKKKRKV

SEQ ID NO: 20 - protein, 1592 aa, artificial sequence
  ABEMaxPI3
MKRTADGSEFESPKKKRKVGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGGSGGSGGSSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNAKTGAAGSLMDVLHYPGMNHRVEITEGILADECAALLCYFFRMPRQVFNAQKKAQSSTDGGSGGSGGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSKRTADGSEFESPKKKRKV

SEQ ID NO: 21 - protein, 1804 aa, artificial sequence
  ABEMax
MKRTADGSEFESPKKKRKVSEVEFSHEYWMRHALTLAKRAWDEREVPVGAVLVHNNRVIGEGWNRPIGRHDPTAHAEIMALRQGGLVMQNYRLIDATLYVTLEPCVMCAGAMIHSRIGRVVFGARDAKTGAAGSLMDVLHHPGMNHRVEITEGILADECAALLSDFFRMRRQEIKAQKKAQSSTDSGGSSGGSSGSETPGTSESATPESSGGSSGGSSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNAKTGAAGSLMDVLHYPGMNHRVEITEGILADECAALLCYFFRMPRQVFNAQKKAQSSTDSGGSSGGSSGSETPGTSESATPESSGGSSGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSKRTADGSEFESPKKKRKV

SEQ ID NO: 22 - protein, 1804 aa, artificial sequence
  dABEMax
MKRTADGSEFESPKKKRKVSEVEFSHEYWMRHALTLAKRAWDEREVPVGAVLVHNNRVIGEGWNRPIGRHDPTAHAEIMALRQGGLVMQNYRLIDATLYVTLEPCVMCAGAMIHSRIGRVVFGARDAKTGAAGSLMDVLHHPGMNHRVEITEGILADECAALLSDFFRMRRQEIKAQKKAQSSTDSGGSSGGSSGSETPGTSESATPESSGGSSGGSSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNAKTGAAGSLMDVLHYPGMNHRVEITEGILADECAALLCYFFRMPRQVFNAQKKAQSSTDSGGSSGGSSGSETPGTSESATPESSGGSSGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSKRTADGSEFESPKKKRKV

SEQ ID NO: 23 - protein, 1660 aa, artificial sequence
  ABEmax?HNH
MKRTADGSEFESPKKKRKVSEVEFSHEYWMRHALTLAKRAWDEREVPVGAVLVHNNRVIGEGWNRPIGRHDPTAHAEIMALRQGGLVMQNYRLIDATLYVTLEPCVMCAGAMIHSRIGRVVFGARDAKTGAAGSLMDVLHHPGMNHRVEITEGILADECAALLSDFFRMRRQEIKAQKKAQSSTDSGGSSGGSSGSETPGTSESATPESSGGSSGGSSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNAKTGAAGSLMDVLHYPGMNHRVEITEGILADECAALLCYFFRMPRQVFNAQKKAQSSTDSGGSSGGSSGSETPGTSESATPESSGGSSGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQGGSGGSRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSKRTADGSEFESPKKKRKV

SEQ ID NO: 24 - protein, 18 aa, artificial sequence
  NLS
MKRTADGSEFEPKKKRKV

SEQ ID NO: 25 - protein, 17 aa, artificial sequence
  NLS
KRTADGSEFEPKKKRKV

SEQ ID NO: 26 - protein, 167 aa, E. coli
MSEVEFSHEYWMRHALTLAKRARDEREVPVGAVLVHNNRVIGEGWNRPIGRHDPTAHAEIMALRQGGLVMQNYRLIDATLYVTLEPCVMCAGAMIHSRIGRVVFGARDAKTGAAGSLMDVLHHPGMNHRVEITEGILADECAALLSDFFRMRRQEIKAQKKAQSSTD